(19) United States
(12) Patent Application Publication
(10) Pub. No.: US 2025/0213860 A1
(43) Pub. Date: Jul. 3, 2025

(54) DEVICE AND METHOD FOR DEBRIDING TISSUE

(71) Applicants: NATIONAL UNIVERSITY OF SINGAPORE, SINGAPORE (SG); NATIONAL UNIVERSITY HOSPITAL (SINGAPORE) PTE LTD, Singapore (SG)

(72) Inventors: Pradeep Paul PANENGAD, Singapore (SG); Unnikrishnan UNNIYAMPURATH, Singapore (SG); Linfa WANG, Singapore (SG); Rajesh Babu DHARMARAJ, Singapore (SG); Ajay Purushothaman NAMBIAR, Singapore (SG); Senthil Kumar ANANTHARAJAN, Singapore (SG); Manoj Krishnan NADUPARAMBIL, Singapore (SG)

(21) Appl. No.: 18/867,143

(22) PCT Filed: May 23, 2023
(86) PCT No.: PCT/SG2023/050356

§ 371 Date: Nov. 19, 2024

(30) Foreign Application Priority Data
May 24, 2022 (SG) 10202205547V
May 24, 2022 (SG) 10202205555T

(51) Int. Cl. A61N 1/32; A61N 1/04; A61N 1/20

(52) U.S. Cl. CPC A61N 1/325; A61N 1/0476; A61N 1/205

(57) ABSTRACT

A device for debriding tissue is described. In an embodiment, the device comprises an electrolysis unit 300 having two electrodes 306, 310. The electrolysis unit 300 is configured to receive an electrolyte for electrically connecting the two electrodes 306, 310. The two electrodes 306, 310 are adapted to connect to a power supply 314 to receive an electric current for electrolysis of the electrolyte, where in use, one of the two electrodes 306, 310 is adapted to provide an acidic region or an alkaline region to the tissue during electrolysis of the electrolyte for debriding the tissue. A method for debriding tissue using the aforementioned device is also described.

### TECHNICAL FIELD

The present disclosure relates to a device and a method for debriding tissue.

### BACKGROUND

Diabetes is a very common disease. For example, about 1 in 9 Singaporeans have diabetes in Singapore, and in a global scale, there are about 537 million people living with diabetes in the world as of 2021. Chronic diabetic foot ulcer is a common problem for diabetic patients. A foot sole of a diabetic patient may thicken easily and dry out to become brittle. This results in cracks or fissures which can be easily contaminated and in turn, develop into chronic foot ulcers. About 6.3% of the diabetic population experience diabetic foot ulcer.

Regular exfoliation and foot hygiene are important for preventing common foot problems like cracks, callosities and fissures which may eventually precipitate into foot ulcers for diabetic patients. Existing devices and/or methods for foot exfoliation include foot masks, manual or electrical foot files, and foot care services (e.g. foot spa or pedicure) which require either performance by a skilled or trained professional, or are slow and unpredictable. For example, a foot spa or a pedicure is expensive and requires pre-booked appointments made with qualified professionals, while use of a foot file may result in skin debris which can attract microbial growth on the foot in a moist environment. Further, use of such foot files, whether manual or electrical, are laborious and are particularly difficult for diabetic patients who are old and/or overweight.

In addition to preventing foot problems through foot hygiene, a device and/or method for attending to wounds, arising for example from such chronic foot ulcers, are also important for the well-being of a patient. The current wound debridement practices, although evolved over the years, are still far from achieving their potential efficiencies. Many of these wound debridement methods still require complex procedures which involve longer and more frequent hospitals visits, and/or higher medical expenditures. This inadvertently affects a quality of life, work productivity and an income or savings of a patient. The situation is worse in chronic debilitating cases including diabetic wounds which may result in amputations.

It is therefore desirable to provide a device and a method for debriding tissue which address the above problems and/or provide a useful alternative.

Furthermore, other desirable features and characteristics will become apparent from the subsequent detailed description and the appended claims, taken in conjunction with the accompanying drawings and this background of the disclosure.

### SUMMARY

Aspects of the present application relate to a device and method for debriding tissue. The tissue may include skin tissue or wound tissue or a wound.

In accordance with a first aspect, there is provided a device for debriding tissue, the device comprising: an electrolysis unit having two electrodes, the electrolysis unit being configured to receive an electrolyte for electrically connecting the two electrodes, the two electrodes are adapted to connect to a power supply to receive an electric current for electrolysis of the electrolyte, wherein in use, at least one of the two electrodes is adapted to provide an acidic region or an alkaline region to the tissue during electrolysis of the electrolyte for debriding the tissue.

Thus, the described embodiment provides a device for debriding tissue. Particularly, by including an electrolysis unit having two electrodes where one of the electrodes is adapted to provide an acidic or an alkaline region to the tissue during electrolysis of the electrolyte, the device is adapted to provide the acidic or the alkaline region to contact the tissue for debriding the tissue. There are numerous advantages with this approach. First, the current received by the electrolysis unit can be controlled to provide a regulated pH environment for the acidic region or the alkaline region for debriding the tissue. This provides a systematic way to control a rate of tissue debridement etc. depending on a condition of the tissue to be debrided. Second, by using the acidic region or the alkaline region for debriding the tissue, it minimizes manual or mechanical placement or movement of the tissue debridement device for debriding the tissue (e.g. in the case of a manual or electronic foot file), thereby providing much convenience to patients who are either old, debilitated, obese or have joint problems by enabling them to perform tissue debridement on their own. Third, in relation to being able to perform tissue debridement independently, this reduces a number of hospital visits by patients which require regular tissue debridement treatment, thereby improving a quality of life for these patients.

Fourth, by using the acidic region or the alkaline region for debriding the tissue by tissue-lysis, the tissue debridement is typically more effective and faster, thereby shortening the time spent by the patients on tissue debridement treatment. Fifth, during electrolysis, electrical charges on the two electrodes allow formation of an ion rich hydration layer on each of the electrode surfaces. Particularly, ions of the same charge and/or larger particles (Bacteria, microbes, and biological macromolecules) with low charge densities are repelled away from the electrode surfaces, thereby providing a non-fouling effect on the electrode surfaces which enables the electrode surfaces to not adhere to a wound surface. This reduces the pain experienced by the patient during opening and examination of the wound. Sixth, ease of use of the device in off-clinical settings also means that it can be used more regularly by users for foot exfoliation or wound debridement as necessary. This helps to reduce delay and reduce e.g. diabetic foot conditions, contributing to a reduction in healthcare costs.

The one of the two electrodes may be perforated. The one of the two electrodes may include a wire mesh or a conductive felt (i.e. an electrically-conductive felt e.g. a graphite felt). The another one of the two electrodes may be grounded. Where the one of the two electrodes is perforated or includes a wire mesh, the one of the two electrodes may be adapted to allow ions having a same polarity as the one of the two electrodes to pass through to an outer side of the electrolysis unit during electrolysis of the electrolyte to provide the acidic region or the alkaline region for debriding the tissue.

The electrolysis unit may comprise an encapsulation made of an electrically insulating material for receiving the electrolyte. The encapsulation may have a window for exposing the one of the two electrodes to the tissue for providing the acidic or alkaline region during electrolysis of the electrolyte.

The device may comprise the electrolyte received by the electrolysis unit.

The electrolyte may be water-based, and the electrolysis unit may comprise an absorbent material for absorbing the electrolyte. Where the electrolyte is water-based, the one of the two electrodes may be adapted to form an ion-rich hydration layer on an electrode surface of the one of the two electrodes during electrolysis of the electrolyte, the ion-rich hydration layer being adapted to repel ions having a same polarity as the one of the two electrodes away from the electrode surface, or particles having a mass equal to or less than 1000 Daltons (Da) and carrying at least an elementary charge (i.e. 1.602×10⁻¹⁹ C) away from the electrode surface. The ion-rich hydration layer may be adapted to repel particles having low charge densities (e.g. bacteria, microbes, and/or biological macromolecules).

The electrolyte may include an organic salt or an inorganic salt, the organic salt or the inorganic salt may have a concentration of between 1 molar to 5 molars. The electrolyte may include a solvent, a salt, a humectant, biological molecules, a surfactant, a wetting agent, an antacid, a pH buffering formulation, an anti-foaming agent, a conditioner compound, an antibiotic, an antimicrobial, a metal chelating compound, a coloring compound or a hydrophobic compound.

Where the one of the two electrodes is a cathode, the cathode may be adapted to provide the alkaline region having a pH level of 9 or above during electrolysis of the electrolyte for debriding the tissue.

The device may comprise the power supply, the power supply may include one of: a DC power supply, a battery or an AC-DC adaptor connected to an AC power supply. Where the power supply includes a battery, the battery may be integrated with the device. The power supply may include a mechanism for reversing a polarity of the electric current.

The power supply may be configured to provide the electric current having a magnitude of less than or equal to 5 A or provide the electric current having a magnitude of less than or equal to 1 A.

The device may comprise a tissue condition sensor configured to detect or monitor a condition of the tissue prior to receiving the electric current for electrolysis of the electrolyte or during the electrolysis of the electrolyte. This helps to ensure that an appropriate duration for the treatment may be set or that a condition of the tissue can be monitored during the tissue debridement process.

The device may comprise a treatment end-point sensor configured to determine an end-point for debriding the tissue. This ensures that the tissue debridement process can be terminated appropriately and to prevent otherwise healthy or live tissue from exposing to the tissue debridement process.

The device may comprise a performance sensor configured to assess a performance of the device for debriding the tissue. This helps to monitor a performance or an output of the device so that elements used in the device (e.g. the electrodes and/or the electrolyte) may be replaced or maintained if necessary.

The device may comprise a pH indicator configured to detect a pH of the acidic region or the alkaline region in contact with the tissue for debriding the tissue. This allows the debridement process to be monitored in-situ.

The device may comprise a vibrator, a rotator, an irrigator or an electrolyte infuser, or a pressure management system adapted to vary a fluid pressure of the electrolyte between the two electrodes. The vibrator and/or the rotator may be configured to provide mechanical friction (e.g. by vibration or rotation) between a surface of the electrode and a surface of the tissue being treated, while the irrigator or the electrolyte infuser, either manual or electronic in nature, may be configured to provide irrigation of electrolyte from a reservoir either to sustain electrolysis or to wash away a debriding surface of the tissue. The pressure management system may work together with the irrigator or the electrolyte infuser to control a fluid pressure (positive or negative) of the electrolyte.

The device may comprise an inflatable bladder attached to the electrolysis unit at an opposite side to the one of the two electrodes, the inflatable bladder may be configured to inflate so as to apply pressure on the electrolysis unit for engaging the one of the two electrodes on the tissue when the device is secured on the tissue. This helps to ensure that the one of the two electrodes is in contact with the tissue during treatment of the tissue. Although it is termed “bladder” in the present disclosure, it should be appreciated that any inflatable device may be attached to the electrolysis unit at the opposite side to the one of the two electrodes for applying pressure on the electrolysis unit when the device is secured on the tissue to be treated.

In accordance with a second aspect, there is provided a kit of parts arranged to be assembled to form any one of the aforementioned devices. The kit of parts may comprise a graphite sheet (e.g. a flexible graphite sheet), a conductive porous sheet (e.g. a steel wire mesh) for use as the electrodes of the electrolysis unit. In an embodiment, the kit of parts includes a non-conductive material (e.g. a non-conductive absorbent material such as a cotton band aid) for use as a spacer between the electrodes of the electrolysis unit.

In accordance with a third aspect, there is provided a method for debriding tissue using a device, the device comprising an electrolysis unit having two electrodes, the electrolysis unit is configured to receive an electrolyte for electrically connecting the two electrodes, the two electrodes are adapted to connect to a power supply to receive an electric current for electrolysis of the electrolyte, wherein one of the two electrodes is adapted to provide an acidic region or an alkaline region to the tissue during electrolysis of the electrolyte for debriding the tissue, the method comprising: (i) providing the electrolyte to the electrolysis unit; (ii) putting the one of the two electrodes in contact with the tissue; (iii) connecting the two electrodes to the power supply to receive the electric current for electrolysis of the electrolyte to provide the acidic region or the alkaline region to the tissue for debriding the tissue.

The power supply may be configured to provide an electric potential difference of less than 20 V between the two electrodes, or to provide an electric potential difference of less than 5 V between the two electrodes. The method may comprise grounding another one of the two electrodes.

The method may comprise controlling the electric current to regulate a pH of the acidic region or the alkaline region for debriding the tissue.

Where the one of the two electrodes is a cathode, the method may comprise controlling the electric current to provide the alkaline region having a pH level of 9 or above at the cathode during electrolysis of the electrolyte for debriding the tissue.

Putting the one of the two electrodes in contact with the tissue may include securing the device to the tissue using a wound dressing or an adhesive bandage, or generating a suction force at the one of the two electrodes by applying a negative fluid pressure on the electrolyte. The negative fluid pressure may be applied using the aforementioned pressure management system.

The device may comprise an inflatable bladder attached to the electrolysis unit at an opposite side to the one of the two electrodes, and the method may comprise inflating the inflatable bladder to apply pressure on the electrolysis unit for engaging the one of the two electrodes on the tissue when the device is secured to the tissue. This helps to ensure that the one of the two electrodes is in contact with the tissue during treatment of the tissue.

The method may comprise providing a solvent, a salt, a humectant, biological molecules, a surfactant, a wetting agent, an antacid, an anti-foaming agent, a pH buffering formulation, a conditioner compound, an antibiotic, an antimicrobial, a metal chelating compound, a coloring compound or a hydrophobic compound in the electrolyte.

Putting the one of the two electrodes in contact with the tissue may expose the tissue to the acidic or alkaline region during electrolysis of the electrolyte for relaxing a network of the tissue so as to allow permeation of the humectant, the biological molecules, the surfactant, the wetting agent, the antacid, the anti-foaming agent, the pH buffering formulation, the conditioner compound, the antibiotic, the antimicrobial, the metal chelating compound, the coloring compound or the hydrophobic compound to penetrate into the tissue.

Embodiments therefore provide a device and method for debriding tissue. Particularly, by including an electrolysis unit having two electrodes where one of the electrodes is adapted to provide an acidic or an alkaline region to the tissue during electrolysis of the electrolyte, the device is adapted to provide the acidic or the alkaline region to contact the tissue for debriding the tissue. First, the acidic region of the alkaline region for debriding the tissue is provided through electrolysis of the electrolyte which is controlled by the current received by the electrolysis unit. This provides a handle for regulating a pH of the acidic region or the alkaline region for controlling the tissue debridement process. Second, tissue debridement using the acidic region or the alkaline region minimizes the need for manual or mechanical placement or movement of a tissue debridement device, e.g. a manual or electronic foot file, for debriding the tissue. This provides much convenience to patients who are either old, debilitated, obese or have joint problems by allowing them to perform tissue debridement on their own. Third, by using the acidic region or the alkaline region for debriding the tissue by tissue-lysis, the tissue debridement is typically more effective and faster, thereby shortening the time spent by the patients on tissue debridement treatment. Fourth, during electrolysis, electrical charges on the two electrodes allow formation of an ion rich hydration layer on each of the electrode surfaces. Particularly, ions of the same charge and/or larger particles (Bacteria, microbes, and biological macromolecules) with low charge densities are repelled away from the electrode surfaces, thereby providing a non-fouling effect on the electrode surfaces which enables the electrode surfaces to not adhere to a wound surface. This reduces the pain experienced by the patient during opening and examination of the wound. Fifth, ease of use of the device in off-clinical settings also means that it can be used more regularly by users for foot exfoliation or wound debridement as necessary. This helps to delay and/or reduce e.g. diabetic foot conditions, thus contributing to a reduction in healthcare costs. Sixth, the device can also be made portable by having integrated battery and this allows the patients to be ambulant while performing the tissue debridement treatment, this improves the quality of life for patients who may require such tissue debridement treatment on a regular basis.

### BRIEF DESCRIPTION OF THE DRAWINGS

Embodiments will now be described, by way of example only, with reference to the following drawings, in which:

FIGS. 1A and 1B show schematic diagrams for electrolysis of water, where FIG. 1A shows a set-up of an electrolytic cell and FIG. 1B shows a zoomed-out diagram of the electrolysis in the electrolytic cell;

FIG. 2 shows a schematic diagram of the electrolysis of water with perforated/porous electrodes in accordance with an embodiment;

FIG. 3 shows a schematic diagram of a modified electrolysis unit in accordance with an embodiment;

FIG. 4 shows a photograph of an electrolytic tissue debridement device in accordance with an embodiment;

FIGS. 5A, 5B, 5C and 5D show diagrams illustrating a pH shift at a cathode of an electrolysis unit during electrolysis of an electrolyte in accordance with an embodiment, where FIG. 5A shows a diagram illustrating electrolysis of a water-based electrolyte, FIG. 5B shows a diagram illustrating a high pH of 14 on a pH strip placed in contact with an active cathode, FIG. 5C shows a diagram illustrating a pH of 9 when a spacer tissue paper is placed between the pH strip and the active cathode, and FIG. 5D shows a diagram illustrating a reduction in the pH level at the cathode when a power supply to the electrolysis unit is switched off;

FIGS. 6A, 6B, 6C, 6D, 6E and 6F show diagrams illustrating a flexible electrode assembly and a pH effect of electrolysis of an electrolyte using the assembled electrode assembly in accordance with an embodiment, where FIG. 6A shows a diagram illustrating an anode and a cathode of the electrode assembly, FIG. 6B shows a diagram illustrating different layers of the electrode assembly, FIG. 6C shows a diagram of the assembled electrode assembly, FIG. 6D shows a diagram of the assembled electrode assembly soaked in neutral pH electrolyte and is placed over pH test strips, FIG. 6E shows a diagram of the assembled electrode assembly of FIG. 6D with power supplied and incubated on the pH strips for a few seconds, and FIG. 6F shows a diagram illustrating a change in colour of the pH test strips in response to a raised pH at a wire mesh surface of the cathode of the assembled electrode assembly which indicates a dynamic pH shift from 7 to 14;

FIGS. 7A, 7B, 7C and 7D show diagrams illustrating an assembly of a device for debriding tissue in accordance with an embodiment, where FIG. 7A shows a diagram of materials used in assembling the device, FIG. 7B shows a diagram illustrating two electrodes (e.g. a cathode and an anode) formed using the materials of FIG. 7A with leads connected and their edges insulated, FIG. 7C shows a diagram illustrating the anode of FIG. 7B being wrapped in a cotton band aid, and FIG. 7D shows a diagram illustrating an assembled device where the electrodes are stacked and stitched together along the edges of the electrodes;

FIG. 8 shows a schematic of a tissue debridement device in accordance with an embodiment;

FIGS. 9A, 9B, 9C and 9D show diagrams illustrating an experiment performed on a pig's skin using the assembled device of FIG. 7D in accordance with an embodiment, where FIG. 9A shows a diagram illustrating the assembled device, FIG. 9B shows a diagram illustrating an ex-vivo pigskin tissue, FIG. 9C shows a diagram illustrating the assembled device attached to the ex-vivo pigskin tissue of FIG. 9B and being activated for a fixed time, and FIG. 9D shows a diagram illustrating a result of the controlled exfoliation produced at the site of the ex-vivo pigskin tissue treated by the assembled device of FIG. 9C as demonstrated by the translucency of the treated area;

FIGS. 10A, 10B and 10C show diagrams illustrating an experiment performed on a pig's sole using the assembled device of FIG. 9A in accordance with an embodiment, where FIG. 10A shows a diagram illustrating a front view and a bottom view of an ex-vivo pig's foot, FIG. 10B shows a diagram illustrating the assembled device used to treat the sole of the ex-vivo pig's foot, and FIG. 10C shows a diagram illustrating a histology of the sole of the ex-vivo pig's foot before and after treatment by the device of FIG. 10B;

FIGS. 11A, 11B and 11C show diagrams illustrating modifications made to the device of FIG. 7D where a bladder is attached to a back side of the device in accordance with an embodiment, where FIG. 11A shows a diagram illustrating a front side of the modified device adapted to be in contact with the tissue for debridement, FIG. 11B shows a diagram illustrating a back side of the modified device on which the bladder is attached, and FIG. 11C shows a diagram illustrating a side view of the modified device;

FIGS. 12A, 12B, 12C, 12D and 12E show diagrams illustrating steps for debriding wound tissue using the modified device of FIG. 11A in accordance with an embodiment, where FIG. 12A shows a diagram illustrating a hypothetical wound, FIG. 12B shows a diagram illustrating the use of an insulator sheath with a cut window placed over the hypothetical wound to expose the wound to be debrided, FIG. 12C shows a diagram illustrating an application of an electrolyte gel on an electrode of the modified device of FIG. 11A, FIG. 12D shows a diagram illustrating stabilisation of the modified device over the wound using a cotton band-aid and FIG. 12E shows a diagram illustrating inflation of the bladder of the modified device for applying pressure on the electrode of the modified device to contact a surface of the wound;

FIG. 13 shows a schematic of a potentiometer circuit for providing a ground or zero potential at one of the electrodes of an electrolytic tissue debridement device in a battery powered configuration in accordance with an embodiment;

FIG. 14 shows an optical micrograph of a porcine dermal tissue after surface treatment using the assembled device of FIG. 11A in accordance with an embodiment;

FIGS. 15A and 15B show micrographs of a surface portion of the porcine dermal tissue of FIG. 14 after surface treatment in accordance with an embodiment, where FIG. 15A shows an optical micrograph of the surface portion with Haematoxylin & Eosin (H&E) staining and FIG. 15B shows a micrograph of the surface portion obtained using scanning electron microscopy (SEM);

FIGS. 16A and 16B show micrographs of an unexposed portion of the porcine dermal tissue of FIG. 14 after surface treatment in accordance with an embodiment, where FIG. 16A shows an optical micrograph of the unexposed portion with Haematoxylin & Eosin (H&E) staining and FIG. 16B shows a micrograph of the unexposed portion obtained using scanning electron microscopy (SEM);

FIG. 17 shows micrographs obtained using digital microscopy to illustrate a thickness of the porcine dermal tissue of FIG. 14 before and after surface treatment in accordance with an embodiment;

FIG. 18 shows photographs of an agar culture and a wire mesh electrode to illustrate smearing of the agar culture on the wire mesh electrode surface in accordance with an embodiment;

FIGS. 19A and 19B show micrographs obtained using scanning electron microscopy (SEM) in relation to the agar culture of FIG. 18, where FIG. 19A shows a micrograph of the agar culture and FIG. 19B shows a micrograph of the agar culture smeared on the wire mesh electrode surface; and

FIGS. 20A and 20B show micrographs obtained using scanning electron microscopy (SEM) after surface treatments of the surfaces smeared with the agar culture of FIG. 18 in accordance with embodiments, where FIG. 20A shows a micrograph of an agar culture-smeared surface after treating with a surfactant comprising 10% sodium dodecyl sulfate (SDS), and FIG. 20B shows a micrograph of an agar culture-smeared surface after the surface is electrolysed as a cathode in a saturated sodium bicarbonate solution.

### DETAILED DESCRIPTION

Exemplary embodiments relating to a device and method for debriding tissue are described.

The device of the present disclosure is a highly effective, easy to use, cost-effective, and safe device for tissue debridement. The device may be used by customers, cosmetic experts, clinicians or podiatrist for performing tissue debridement. In the present disclosure, tissue debridement means removal of unwanted tissues, for example, dead tissue, injured nonviable tissue (crushed tissue), contaminated tissue, infected tissue, pathological tissue or wound tissue. Examples of potential applications for tissue debridement include: 1) skin exfoliation or skin debridement of the heel and/or sole of the foot (for example for diabetic patients to delay or prevent foot ulceration or “diabetic foot”), and 2) wound debridement (for example, for diabetic or other ulcers). Regular exfoliation and foot hygiene, and wound debridement are important in preventing common foot problems like cracks, callosities, dry foot, fissures and/or complications of diabetic foot ulcer.

The present disclosure exploits an acidic environment around the anode or an alkaline environment around the cathode in an electrolysis unit to produce a controlled and precise tissue-lysis or tissue disintegration for effecting tissue debridement. It was found that exposing tissue to a non-neutral pH environment (i.e. pH away from pH=7, e.g. an acidic or an alkaline environment) is able to remove or debride tissue, particularly unwanted tissue or infected tissue. In the embodiments described below, the alkaline environment around the cathode in the electrolysis unit was exposed to the tissue to effect the tissue debridement. In an embodiment, the electrolysis unit is compacted into a disposable (e.g. single use and/or recycled) flat flexible skin treating/wound dressing electronic patch. In an embodiment, the device for debriding tissue includes an electrolysis unit which has an active cathode surface. The active cathode surface provides a high alkaline pH region, during electrolysis of an electrolyte, to the tissue to be treated or debrided. The high pH micro-zone or region generated on a skin surface or a wound surface results in controlled tissue disintegration, thereby producing exfoliation of dead and flaky outer layers of the skin or exfoliation of dead tissue layers on the wound surface. As will be described below in relation with the Figures, the device was first tested on ex-vivo pigskin and soft tissue samples. A device was then tested on an in-vitro pig-foot sole and porcine dermal tissue to demonstrate its efficiency in skin exfoliation with histopathological evidences. The device was further modified and was used on a hypothetical wound to demonstrate use of the device as a wound debridement device.

### An Electrolytic Tissue Debridement Device

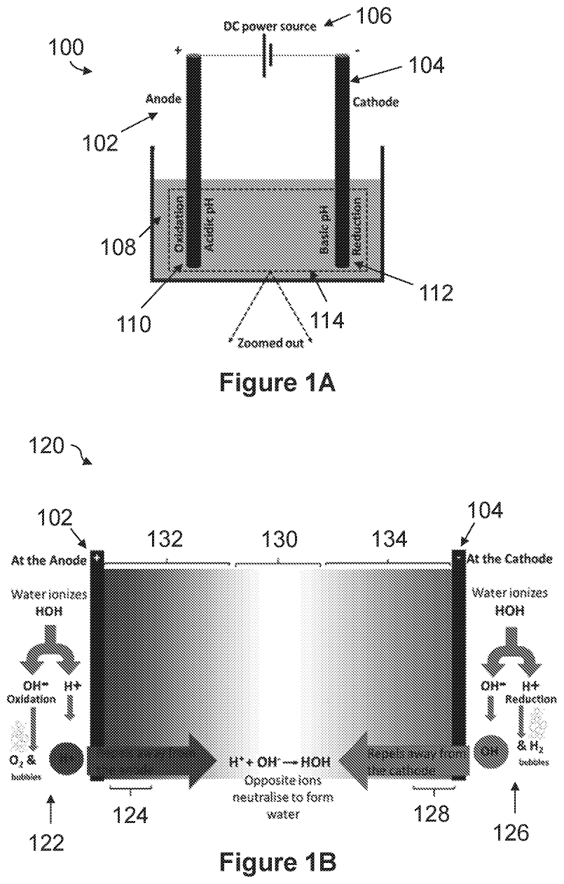

An electrolytic tissue debridement device was developed based on the principles of electrolysis. The electrolytic tissue debridement device of the present embodiment uses either an acidic micro-zone tapped out from an anode surface or a basic/alkaline micro-zone tapped out from a cathode surface, of a compacted modified electrolysis unit. This is shown in relation to FIGS. 1A to 4.

FIGS. 1A and 1B show schematic diagrams for the electrolysis of water, where FIG. 1A shows a set-up 100 of the electrolytic cell and FIG. 1B shows a zoomed-out diagram 120 of the electrolysis in the electrolytic cell.

As shown in FIG. 1A, the electrolytic cell includes an anode 102 and a cathode 104 which are connected to a direct current (DC) power source 106. During electrolysis when a current is passed through the anode 102 and the cathode 104 via an electrolyte 108 (e.g. having a salt in water), an oxidizing and acidic region 110 around the anode 102 (positive electrode), and a reducing and basic region 112 around the cathode 104 (negative electrode) are produced. A portion 114 of the electrolytic cell is zoomed-out to illustrate the electrolysis in the electrolytic cell and this is shown in FIG. 1B.

Referring to FIG. 1B and as described above, when the DC power source 106 of sufficient power is connected to electrodes 102, 104 immersed in an electrolyte, electrolysis occurs, producing an oxidizing and acidic environment around the anode 102, and a reducing and basic environment around the cathode 104. In the present embodiment where the electrolyte comprises water and a salt, water ionizes into H⁺ and OH⁻ ions at the anode 102 and the cathode 104, respectively. At the anode 102, the OH⁻ ions lose electrons, get oxidized and form oxygen which bubbles out, as illustrated by 122. The remaining H⁺ ions formed are repelled away from the positively charged anode 102, creating an acidic region 124 around the anode 102. As a result, at the anode 102, OH⁻ ions are deducted and H⁺ ions are produced proportional to the current flowing through the electrodes 102, 104.

On the other hand, at the cathode 104, the H⁺ ions receive electrons, get reduced and form hydrogen which bubbles out, as illustrated by 126. The remaining OH⁻ ions formed are repelled away from the negatively charged cathode 104, creating a basic/alkaline region 128 around the cathode. As a result, at the cathode 104, the H⁺ ions are deducted and the OH⁻ ions are produced proportional to the current flowing through the electrodes 102, 104. Hence, the anode 102 and the cathode 104 produce an acidic pH region 124 and a basic pH region 128, respectively, where a magnitude or an intensity of the pH of each of these regions 124, 128 is proportional to the current flowing through the electrodes 102, 104. This provides a handle to control a pH of the acidic region 124 around the anode 102 and/or a pH of the alkaline region 128 of the cathode 104.

It should be appreciated that the acidic and alkaline regions 124, 128 as shown in FIG. 1B are in a dynamic state where the H⁺ ions are being continuously generated at the anode 102 while the OH⁻ ions are being continuously generated at the cathode 104, and therefore maintaining a high concentration of H⁺ ions near the anode 102 and a high concentration of OH⁻ ions near the cathode 104 even though they are constantly being repelled away from the anode 102 and the cathode 104, respectively. Also, as shown in FIG. 1B, as the H⁺ ions and the OH⁻ ions are being repelled towards the center of the electrolytic cell, these H⁺ ions and OH⁻ ions can meet and react to form water. This results in decreasing concentrations of H⁺ ions and OH⁻ ions from the anode 102 and the cathode 104, respectively, and a PH neutral region 130 at or near the center of the electrolysis cell. The acidic pH zone 132 and the basic/alkaline pH zone 134 are also shown in FIG. 1B. It should therefore be appreciated that a pH gradient is formed within the electrolytic cell as the pH increases from the acidic pH zone 132 to the PH neutral region 130 towards the basic/alkaline pH zone 134.

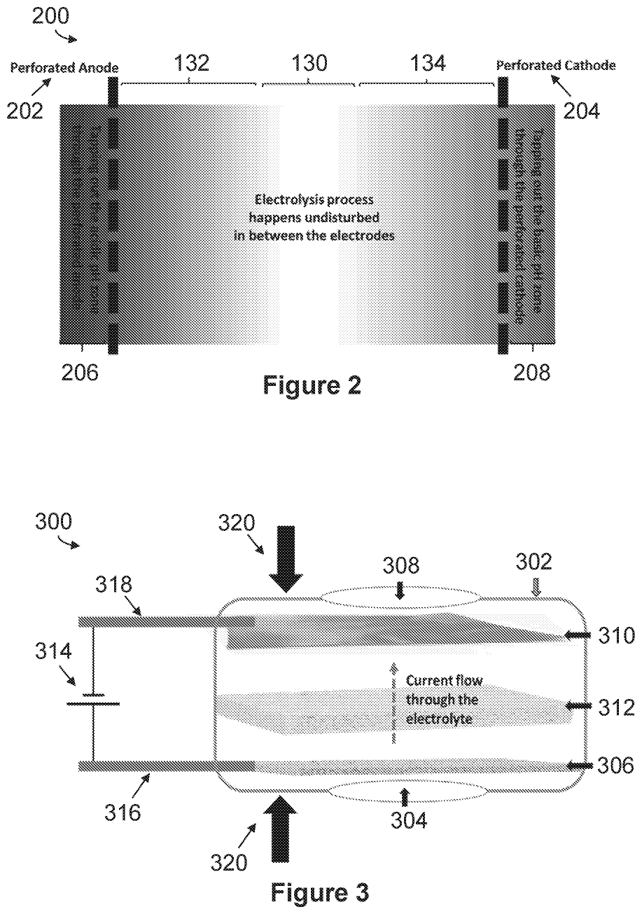

FIG. 2 shows a schematic diagram 200 of the electrolysis of water with perforated/porous electrodes in accordance with an embodiment. As described in relation to FIG. 1B, when the DC power source 106 of sufficient power is connected to the electrodes 102, 104 immersed in an electrolyte (e.g. water with a salt), electrolysis occurs producing the oxidizing and acidic region 110 around the anode 102, and the reducing and basic region 112 around the cathode 104. If the electrodes (the anode 102 and/or the cathode 104) are porous or perforated, the respective pH zones can be tapped out of the electrolysis unit without disturbing the process of electrolysis happening in the electrolyte between the electrodes. FIG. 2 shows a perforated anode 202 and a perforated cathode 204. As the perforated electrodes 202, 204 allow ions having a same polarity as the perforated electrodes 202, 204 to pass through it onto an outer side of the electrolytic cell (i.e. moving from the pH zones 130, 132, 134 (or space) in the electrolyte between the two electrodes 202, 204 towards the ends of the electrolytic cell), this aids the formation of a low pH zone 206 (e.g. for the perforated anode 202) or a high pH zone 208 (e.g. for the perforated cathode 204). The respective pH zones 206, 208, as shown in FIG. 2, can be formed at the extreme ends of the electrolytic cell/electrolysis unit, and at outer sides of the electrolytic cell. In the present embodiment, the pH zones 206, 208 may each has a width of a few hundreds of microns. These pH zones 206, 208 may be termed “extreme pH zones” as they are close to the electrodes 202, 204 and are likely to be either a most acidic region (i.e. lowest in pH) or a most alkaline region (that is highest in pH) of the electrolytic cell.

To illustrate the above, an example using a sodium iodide (NaI) salt dissolved in a water electrolyte is described. In this example, the negatively charged ions are iodide (I⁻) and hydroxyl (OH⁻), while the positively charged ions are sodium (Nat) and hydronium (H⁺) (i.e. NaI ionizes to form Na⁺ ions and I⁻ ions, and H₂O ionizes to form H⁺ ions and OH⁻ ions). In this case, a perforated cathode predominantly allows negatively charged iodide (I⁻) and hydroxyl (OH⁻) to pass through the cathode as the positively charged ions are tightly bound or attracted to the cathode surface. On the other hand, a perforated anode will predominantly allow positively charged sodium (Na⁺) and hydronium (H⁺) ions to pass through the anode as the negatively charged ions are tightly bound or attracted to the anode surface.

This particular characteristic of an electrolysis unit with perforated electrodes allows an amplification of pH at the “extreme pH zones”, thereby providing regions of high concentrations of charged ions for treating/debriding the tissue concerned.

FIG. 3 shows a schematic diagram of a modified electrolysis unit 300 in accordance with an embodiment. As shown in FIG. 3, the modified electrolysis unit 300 is encapsulated with an encapsulation 302 comprising a non-electrolyte permeable and non-conductive material (i.e. a non-electrically conductive material e.g. plastic). In the present embodiment, the encapsulation 302 includes a window 304 formed in the encapsulation 302 at an anode 306 of the modified electrolysis unit 300 to expose a surface of the anode 306 and a window 308 formed in the encapsulation at a cathode 310 of the modified electrolysis unit 300 to expose a surface of the cathode 310. In the present embodiment, windows 304, 308 are provided at both the anode 306 and the cathode 310 of the modified electrolysis unit, but it should be appreciated in other embodiments, a window in the encapsulation can be provided at either the anode or the cathode. With these windows in the encapsulation 302, the high pH zone (i.e. the pH zone at the cathode 310) and/or the low pH zone (i.e. the pH zone at the anode 306) can be accessed. In relation to the above description for FIG. 2, the windows 304, 308 in the encapsulation 302 therefore provide a means to extract or tap out the pH zones from the anode 306 and/or the cathode 310 to be exposed to the tissue to be treated. The encapsulation 302 with the windows 304, 308 of the modified electrolysis unit 300 therefore provides a means for the pH micro-zone to be extracted without the risk of spillage or spread which will cause unwanted tissue injury.

In the present embodiment, the anode 306 can be formed using a flexible graphite felt or a corrosion resistant wire mesh and the cathode 310 can be formed using a metallic fine wire mesh, although it should be appreciated other suitable materials may be applicable. In the present embodiment, a non-conducting material 312 adapted to absorb the electrolyte can be placed between the anode 306 and the cathode 310 within the encapsulation 302. The non-conducting material 312 serves to hold the electrolyte while allowing a current to flow through the electrolyte. The current can be provided to the modified electrolysis unit 300 by a circuit comprising a power supply 314, a positive lead 316 electrically connected to the anode 306 and a negative lead 318 electrically connected to the cathode 310. The modified electrolysis unit 300 can be compacted as shown by the arrows 320 to form a thin flexible tissue debridement device or tissue debridement patch.

Tissues exposed to the acidic or the alkaline environment of such pH zones will undergo disintegration or debridement. This is exploited in designing an electrolytic tissue debridement device as discussed below.

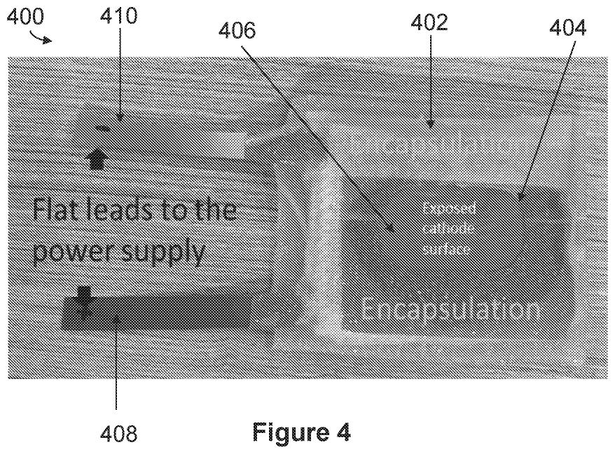

FIG. 4 shows a photograph of an electrolytic tissue debridement device 400 in accordance with an embodiment. As shown in FIG. 4, the electrolytic tissue debridement device 400 includes an encapsulation 402 having a window 404 (e.g. an oval window) on a perforated cathode 406 for tapping out an alkaline environment, for example a high pH micro-zone (e.g. a pH of 9 to 14), to effect tissue disintegration or exfoliation. The perforated cathode 406 in the present embodiment includes a fine steel wire mesh. The electrolytic tissue debridement device 400 can be connected to a programmable power supply (e.g. with or without sensors) using the leads 408, 410 to produce optimum exfoliation or tissue debridement. The electrolytic tissue debridement device 400 as shown in FIG. 4 is in a form of a patch, and it can be secured or fixed to an area of skin tissue or an area of wound tissue to be treated. This may be performed using an adhesive, or other forms of dressing, to keep the electrolytic tissue debridement device 400 in place during the course of the treatment. In the present embodiment, the oval window 404 of the electrolytic tissue debridement device 400 can be secured onto the area of the skin tissue or the area of the wound tissue to be treated to expose the area of the skin tissue or the area of the wound tissue to a high pH environment provided by the perforated cathode 406. Although use of high or extreme pH environments are described above, it should be appreciated that in embodiments, an acidic or an alkaline environment (i.e. a non-neutral pH environment or an environment having a pH less than or more than 7) can also effect tissue debridement. In this electronic debridement device of the present embodiment as explained above, the electric current flowing between the electrodes aids the ionization of the molecules at the electrodes. At the same time, this electric current provides movement of ions (e.g. the H⁺ and OH⁻ ions) which may in turn produce heating at or around the electrodes for aiding the tissue debridement process. In an embodiment, an incubation period or a treatment period can be determined using a treatment period sensor connected to the electrolytic tissue debridement device for determining or calculating the incubation period. The electrolytic tissue debridement device can be designed with different sizes or with different levels of miniaturization or expansion.

Table 1 below provides some benefits or advantages associated with different features of the electrolytic tissue debridement device of the present embodiment.

TABLE 1
Features of the electrolytic tissue debridement device and their benefits

| Feature | Benefit/Advantage |
|---|---|
| Tapping out an extreme pH micro-zone | Extreme pH tissue-lysis to remove dead and |
| on the tissue interacting surface from a | flaky layers from a surface of a skin or a |
| basic electrolysis unit through perforated | wound |
| electrodes (Anode/Cathode) | |
| Extreme pH micro-zone incubation | Swelling and relaxation of the protein |
| | meshwork of the surface layers of the |
| | cornified skin or wound to imbibe water and |
| | other molecules- moisturizing effect |
| Generation of a thin extreme pH micro- | Extreme pH micro-zone that can be |
| zone that can be switched ON & OFF | operated without the risk of spillage/spread, |
| electrically | that causes unwanted tissue injury |
| Generation of a thin extreme pH zone | A controllable extreme pH zone according to |
| that can be switched ON & OFF and | needs by using a programmable power |
| controlled electrically | supply |
| A device design that is cheap and | Single use/disposable/recyclable tissue |
| affordable | interacting pad |
| A fully-electronic device with no need for | Easy to use, not skill dependent |
| mechanical movements | |

Potential commercial applications of the electrolytic tissue debridement device may include: 1) Electronic pedicure-A non-mechanical/non-skill dependent, efficient, finely controlled, safe foot exfoliation device that can be used even by overweight, aged and debilitated patients especially diabetic patients to avoid foot ulceration; 2) Manicure; 3) Facial exfoliation; 4) Removal of callous on the skin; 5) Wound debridement; and 6) Wound bed sterilization with high pH.

### Working of the Electrolytic Tissue Debridement Device

In the present embodiment, the electrolytic tissue debridement device utilizes a high pH on a surface of a cathode in an electrolysis unit. The high pH is used to hydrolyze structural proteins to produce tissue-lysis. As described above, a basic electrolysis unit can be designed in a specific way to achieve the desired function.

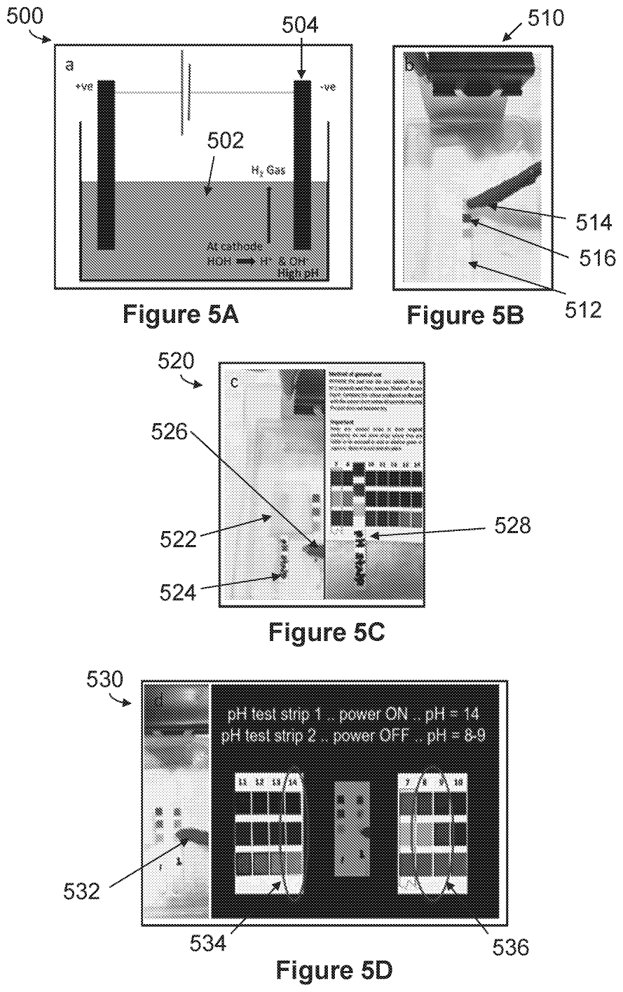

In an electrolysis unit, a very high pH (e.g. a pH of 9 to 14) develops around the cathode. This high pH zone exists only when the power is ‘ON’ and is confined to a thin micro-zone around the cathode. This is illustrated in relation to FIGS. 5A to 5D below. These properties make the cathode an ideal tissue-lysing or tissue disintegrating electrode. This property of the cathode is exploited in the electrolytic tissue debridement device to cause hydrolysis of the unwanted tissues.

FIGS. 5A, 5B, 5C and 5D show diagrams/photographs illustrating pH shift at a cathode of an electrolysis unit during electrolysis of an electrolyte in accordance with an embodiment.

FIG. 5A shows a diagram 500 illustrating electrolysis of a water-based electrolyte 502. As shown in FIG. 5A, during electrolysis of the water-based electrolyte 502, the water is ionized near the cathode 504 (i.e. the negative electrode) into hydronium (H⁺) and hydroxyl (OH) ions. At the cathode 504, the hydronium ions are reduced and escaped as hydrogen gas, leaving behind the hydroxyl ions which raise the pH around the cathode surface. FIG. 5B shows a photograph 510 illustrating a high pH of 14 on a pH strip 512 placed in contact with an active cathode 514. The photograph 510 of FIG. 5B illustrates touching of the pH strip 512 with the active cathode 514 of an electrolysis unit, where the pH strip 512 indicates a high pH of 14 (labelled as 516) upon contact with the cathode surface. FIG. 5C shows a photograph 520 illustrating a reduction of pH to a pH of 9 when a spacer tissue paper 522 is placed between the pH strip 524 and the active cathode 526, as compared to a pH of 14 achieved in relation to FIG. 5B. As shown in FIG. 5C, the spacer tissue paper 522 of 300 micron is used to cover the pH strip 524 when being treated with the cathode 526 as used in FIG. 5B, and the pH strip indicated a pH of 9 in this case (see the right panel 528 of the photograph 520). This shows that the high pH zone produced by the cathode stays confined to a micro-zone around the cathode. This concept can be exploited by including e.g. a thin ion permeable spacer layer between the cathode surface and the tissue to be treated to reduce or control a pH environment (e.g. to lower the pH) around the cathode. FIG. 5D shows a diagram 530 illustrating a reduction in a pH level at the cathode 532 when the power to the electrolysis unit is switched off. As shown in FIG. 5D, switching off the power terminates the high pH activity of the cathode 532 to less than a pH of 9 in a few seconds. This is shown in the right panel of diagram 530 where the pH of a pH test strip 1 indicates a pH of 14 (labelled as 534) when the power to the cathode 532 is turned on, and the pH of a pH test strip 2 indicates a pH of 9 (labelled as 536) when the power to the cathode 532 is turned off. Hence, it can be inferred that the high pH zone around the cathode 532 is confined to a micro-zone which can be extinguished by switching off the power.

Examples of a suitable water-based electrolyte which can be used includes (i) sodium citrate or potassium citrate, (ii) sodium ascorbate or potassium ascorbate, (iii) sodium bicarbonate or potassium bicarbonate, (iv) sodium sulfate or potassium sulfate, or (v) sodium phosphate or potassium phosphate salts dissolved in water. Each of these salts may have different levels of sodium or potassium ions e.g. the salts may include a mono-sodium or mono-potassium, di-sodium or di-potassium, or tri-sodium or tri-potassium salt. It should also be appreciated that in embodiments, solid state electrolytes (SSEs) can also be used if their matrix is water stable and may be flexible. When placed between the electrodes, the solid matrix of these solid-state electrolytes should not cause undesirable reaction between moist tissues being treated with the electrode assembly. In an embodiment, a solid-state electrolyte includes salt hydrates. Examples of a solid hydrated salt which can provide H⁺ and OH⁻ ions include LiClO₄·3H₂O and sodium sulfate·nH₂O. The pH shift in the electrode assembly is caused by ionization of water molecules to H⁺ and OH⁻ ions and removal of one of these ionic species at the electrode surface by reduction or oxidation as described above. The water contributing as the source for H⁺ and OH⁻ ions can either come from the moist tissue being treated or the hydrated salt in the SSE or both.

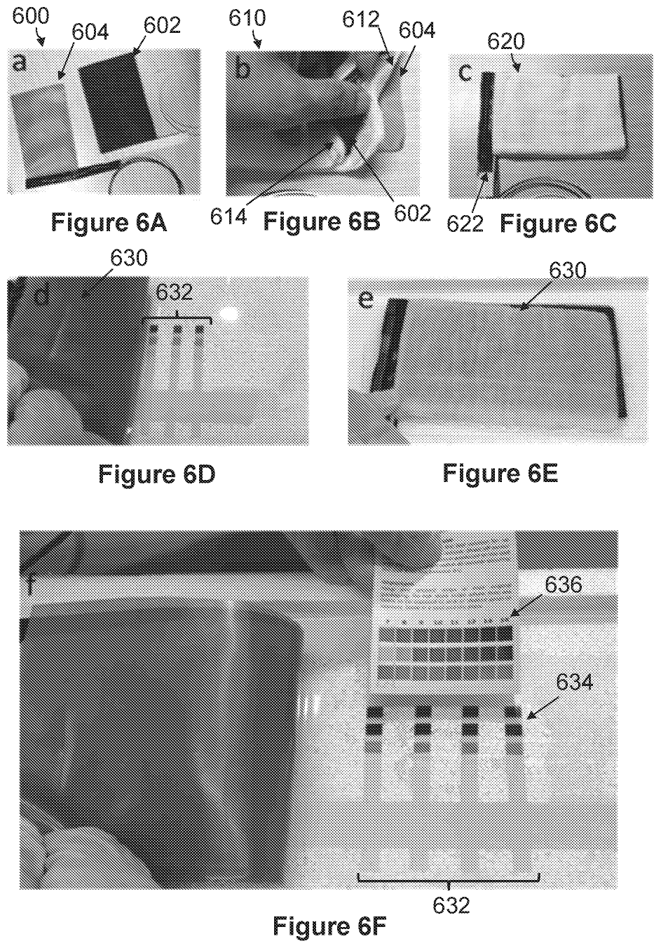

FIGS. 6A, 6B, 6C, 6D, 6E and 6F show photographs illustrating pH effect of electrolysis of an electrolyte using a flexible electrode assembly in accordance with an embodiment. This series of photographs in relation to FIGS. 6A to 6F also show how an electrolysis device can be assembled as a thin flexible assembly, in accordance with an embodiment.

FIG. 6A shows a photograph 600 illustrating an anode 602 and a cathode 604 of the electrode assembly. As shown in FIG. 6A, the electrolysis device in this embodiment includes a flexible anode 602 made of rayon graphite felt and a cathode 604 made of 316 stainless steel wire mesh. It should be appreciated that 316 stainless steel is a type of steel with specific ingredients added to the iron to make the steel alloy mix, and is typically used for medical devices. The number “316” is unrelated to a density of wire mesh or sizing. Each of the anode 602 and the cathode 604 are also connected to leads which can be connected to a power supply.

FIG. 6B shows a photograph 610 illustrating different layers of the electrode assembly. As shown in FIG. 6B, the electrode assembly (or an electrolysis device) includes the anode 602 and the cathode 604 which are separated or covered by a white absorbent gauze 612. The absorbent gauze 612 functions to separate the anode 602 and the cathode 604 of the electrode assembly, while at the same time, to hold an electrolyte between the anode 602 and the cathode 604 for the electrolysis process. The electrode assembly as shown in FIG. 6B has four layers, which are (from bottom to top) a flexible cathode wire mesh 604, the absorbent gauze 612, the flexible anode graphite felt 602 and a superficial absorbent gauze 614.

FIG. 6C shows a photograph of the assembled electrode assembly 620. As shown in FIG. 6C, the electrode assembly 620 includes leads 622 connected to each of the electrodes (i.e. the anode 602 and the cathode 604). The connection points between the leads 622 and the electrodes 602, 604 are isolated with coating for water proofing and electrical isolation. The bare cathode surface at the bottommost end of the electrode assembly 620 functions to come in contact (e.g. direct or indirect contact) with the tissue to be treated. This surface of the bare/exposed cathode 604 is an active surface or a treating surface of the electrode assembly 620.

FIG. 6D shows a photograph of the assembled electrolysis device 630 soaked in neutral pH electrolyte and is placed over pH test strips 632. The assembled electrolysis device 630 is connected to the power supply to allow electrolysis of the electrolyte to occur, and is incubated on the pH strips for a few seconds, as shown in FIG. 6E.

FIG. 6F shows a photograph illustrating a change in colour of the pH test strips 632 in response to the raised pH at the cathode wire mesh surface of the assembled electrolysis device 630 after the incubation of the assembled electrolysis device 630 on the pH strips. As shown in FIG. 6F, the change in colour 634 of the pH strips indicates a dynamic pH shift from 7 (as shown at FIG. 6D) to 14 (as shown in FIG. 6F). The high pH of 14 of the pH strips is indicated on the pH colour code reference sheet 636. This high pH at the cathode wire mesh can induce tissue disintegration and can effect tissue debridement as demonstrated in relation to FIGS. 9A to 10C, and FIGS. 14 to 17 below.

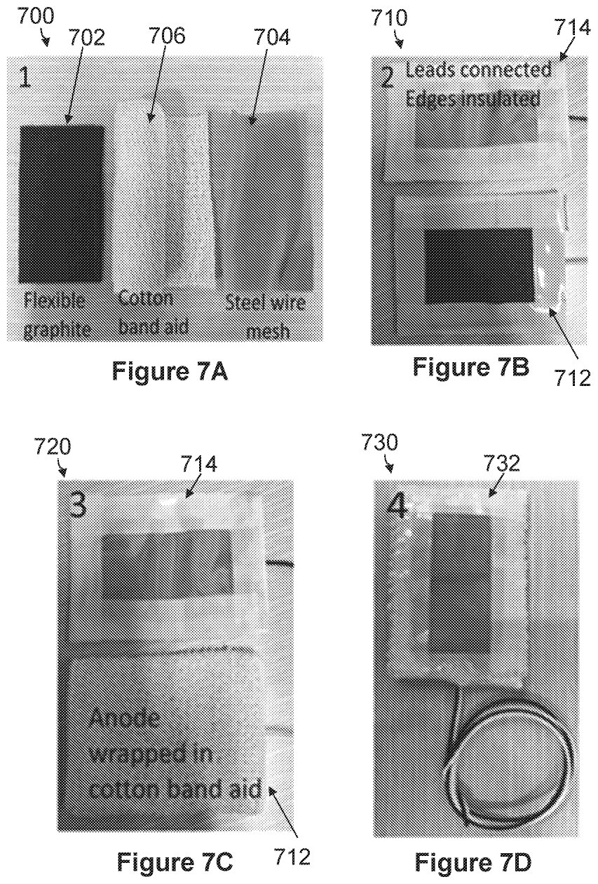

FIGS. 7A, 7B, 7C and 7D show photographs illustrating an assembly of a device for debriding tissue in accordance with an embodiment.

FIG. 7A shows a photograph 700 of materials used in assembling a tissue debridement device in accordance with an embodiment. The materials include a flexible graphite felt 702 for use as an anode, a flexible fine grade steel wire mesh 704 for use as a cathode, and a cotton band-aid 706. FIG. 7B shows a photograph 710 illustrating two electrodes 712, 714 (e.g. the anode 712 and the cathode 714) with leads connected, and their edges and connection points were insulated. FIG. 7C shows a photograph 720 illustrating the anode 712 of FIG. 7B being wrapped in the cotton band aid 706. This is to avoid direct contact (or shorting) between the opposite electrodes (i.e. the anode 712 and the cathode 714). FIG. 7D shows a photograph 730 illustrating an assembled electrolytic device 732 where the cathode 714 is placed over the wrapped anode 712 and trans-stitched together with cotton thread through the insulated borders of the electrodes.

It will therefore be appreciated that a kit of parts for a tissue debridement device can be provided and assembled to form the tissue debridement device 732, as illustrated by FIGS. 7A to 7D in an exemplary embodiment. Basic components of the kit of parts may include an anode, a cathode, and optionally an absorbent material to be sandwiched between the anode and the cathode (e.g. in the case of a water-based electrolyte or electrolyte gel; may not be necessary in the case of a solid electrolyte). Additional components may include the electrolyte, leads connecting to the anode and the cathode, a power supply, and encapsulation (e.g. made of insulating material) of the electrolysis unit. In an embodiment, an ion-permeable and/or water-permeable spacer or layer may also be included. The ion-permeable and/or water-permeable spacer or layer can be placed between the exposed electrode surface and the tissue being treated when the tissue debridement device is assembled. This aids to guard the tissue from extreme pH by reducing a high pH environment on the tissue (e.g. by spacing it further away from the electrode) and/or prevent exposing the tissue to any electric current or leakage current by not having direct contact between the electrode and the tissue to be treated.

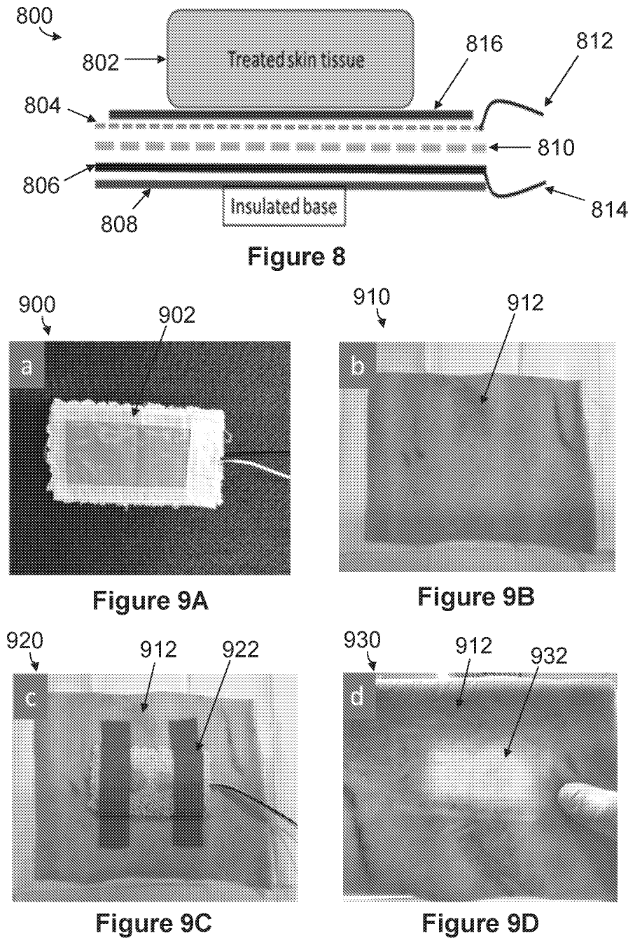

Embodiments of the electrolytic/electrolysis device can be applied and used for tissue debridement, for example, for the purposes of foot exfoliation or wound debridement. These are discussed in relation to FIGS. 8 to 10C below.

### Electrolytic Tissue Debridement Device as a “Foot Exfoliation Device”

A “Foot exfoliation device” can be used as a sole and heel exfoliation patch/pad for achieving de-bulking through exfoliation of a thick cornified layer on a skin surface of a sole or a heel. This can be used in cosmetic applications and/or medical applications.

FIG. 8 shows a schematic of an electrolytic tissue debridement device 800 in accordance with an embodiment, which can be used as a foot exfoliation device. The foot exfoliation device is an electronic device for achieving exfoliation and thinning of the superficial dead skin layer (e.g. stratum corneum) of a skin tissue 802. Similar to the tissue debridement device as afore-described, the foot exfoliation device 800 comprises an electrolysis unit having a perforated cathode 804 (e.g. made of wire mesh) and an anode 806 (e.g. made of a graphite felt) which is attached to an insulated base 808. In the present embodiment, the electrolysis unit also includes an absorbent layer 810 (e.g. an absorbent cotton layer) sandwiched between the perforated cathode 802 and the anode 806 for holding an electrolyte. The device 800 can be connected to a power supply (e.g. a rechargeable battery pack) using a negative lead 812 and a positive lead 814. When the power supply connected to the device 800 is switched on, an electric current passes through the electrolyte to cause electrolysis of the electrolyte. This induces a high pH micro-zone 816 on an upper (or exposed) surface of the cathode 804 of the foot exfoliation device. The alkaline environment of the high pH micro-zone 816 digests and thins down a layer of dead skin at a surface of the skin tissue 802. This high pH micro-zone 816 is formed or induced only when the device is active. In an embodiment, the electrolytic tissue debridement device includes a current control for controlling the current received by the electrodes for controlling a pH of the acidic or the alkaline region. The current may be controlled to have a magnitude of less than or equal to 5 A, or less than or equal to 1 A. An example of a current control is a variable resistor connected to an electric circuit of the electrolytic tissue debridement device.

FIGS. 9A, 9B, 9C and 9D show photographs illustrating an experiment performed on a pig's skin using the assembled device of FIG. 7D in accordance with an embodiment. The electrolytic tissue debridement device is being used as a foot exfoliation device in this case.

FIG. 9A shows a photograph 900 illustrating the assembled device 902 similar to that as shown in FIG. 7D. The electrolytic tissue debridement device was used as a foot exfoliation device and demonstrated using a pig's skin. FIG. 9B shows a photograph 910 illustrating an ex-vivo pigskin tissue 912 used in testing the electrolytic tissue debridement device 902 of FIG. 9A. FIG. 9C shows a photograph 920 illustrating the assembled device 902 being attached onto the ex-vivo pigskin tissue 912 of FIG. 9B using adhesive tape 922 and being activated for a fixed time. FIG. 9D shows a photograph 930 illustrating a result of the controlled exfoliation produced at the site treated by the assembled device 920. As shown in FIG. 9D, a precise and controlled exfoliation was produced at the site treated. This is demonstrated by the translucency of the treated area 932 on the pigskin tissue 912. As shown in FIG. 9D, the treated tissue was positioned over a trans-illuminator.

For the experiment as shown in relation to FIGS. 9A to 9D, the electrolyte used was 1 molar dibasic guanidine citrate/(guanidine) 2 citrate in water. An initial pH (which was near 8) was adjusted to exactly pH 7 by adding a small amount of citric acid to the original 1 molar solution. A voltage of 4 V and a current of 500 mA were used. The current was kept below 1 A to reduce the heating effect on the tissue being treated. The power (i.e. V×I) used was 2 W. The current density over the 20 cm² area of the patch was 25 mA/cm². Current density and ion concentrations were the most important determinants of the pH shift on the cathode surface of the device 902. The device 902 digested pig skin tissue at a rate of 10 microns thick tissue layer per minute. In 10 minutes, approximately 10×10=100 microns thick layer of tissue was removed from the surface of the pigskin tissue 912. The guanidine citrate was used because both guanidine and citric acid are normally present in a human body. Compounds like sodium chloride and potassium chloride should ideally not be used as part of the electrolyte as free chlorine gas (i.e. a toxic gas) may be released at the anode during electrolysis.

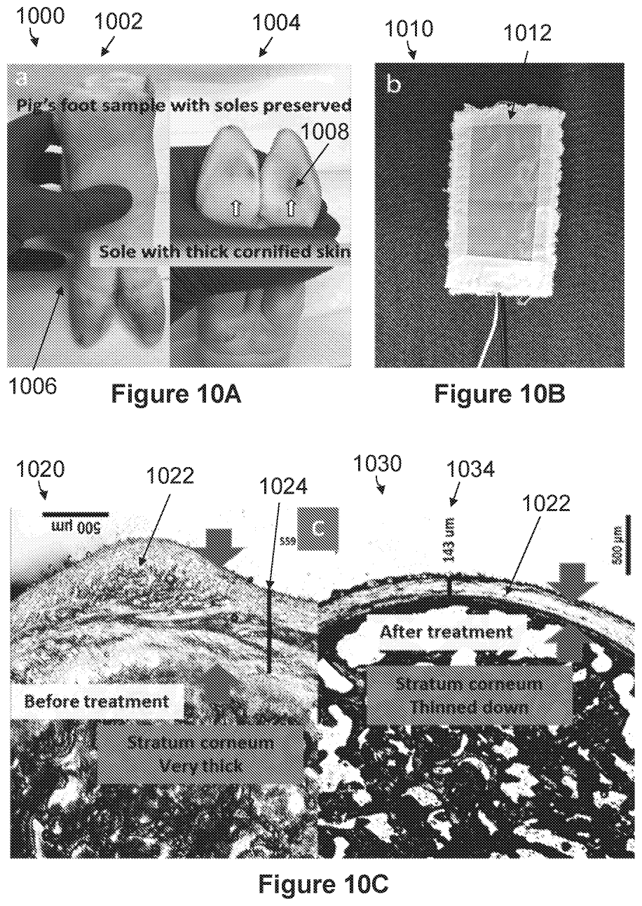

FIGS. 10A, 10B and 10C show photographs or micrographs illustrating an experiment performed on a pig's sole using the assembled device of FIG. 9A in accordance with an embodiment.

FIG. 10A shows a photograph 1000 illustrating a front view 1002 and a bottom view 1004 of an ex-vivo pig's foot sample 1006. The soles of the pig's foot sample 1006 have thick cornified skin 1008. FIG. 10B shows a photograph 1010 illustrating the assembled device 1012 used to treat the sole of the ex-vivo pig's foot sample 1006. The assembled device 1012 as shown in FIG. 10B is similar to that shown in FIG. 9A. The treatment applied was similar to what was described in relation to FIG. 8. FIG. 10C shows two micrographs 1020, 1030 illustrating a histology of the sole of the pig's foot sample 1006. The micrograph 1020 shows the histology of the sole of the pig's foot sample 1006 before the tissue debridement treatment by the assembled device 1012 of FIG. 10B and the micrograph 1030 shows the histology of the sole of the pig's foot sample 1006 after the tissue debridement treatment by the assembled device 1012. As shown in the micrograph 1020, a stratum corneum layer 1022 of the sole of the pig's foot sample 1006 has an initial thickness of 559 μm. After the tissue debridement treatment, the histology of the sole of the pig's foot sample 1006 shows significant thinning of the stratum corneum layer 1022. As shown in the micrograph 1030, the stratum corneum layer 1022 was thinned down to about 143 μm after the tissue debridement treatment. This proves the exfoliative potential of the assembled device 1012 for performing exfoliation of the foot sole.

### Electrolytic Tissue Debridement Device as a “Wound Debridement Device”

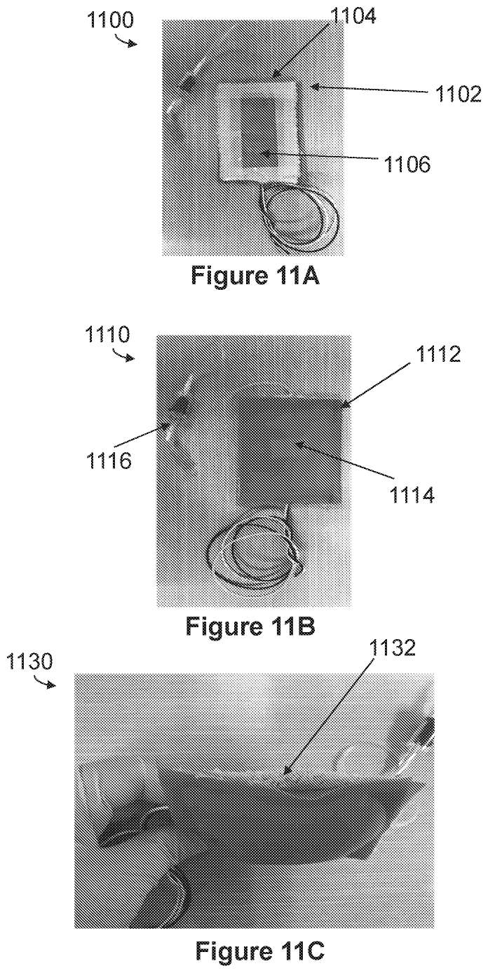

Besides applying the electrolytic tissue debridement device as a “foot exfoliation device”, it can also be used as a “wound debridement device”. FIGS. 11A, 11B and 11C show photographs illustrating modifications made to the electrolytic device 732 of FIG. 7D where a bladder is attached to a back side of the device in accordance with an embodiment.

FIG. 11A shows a photograph 1100 illustrating a front side 1102 of the modified device 1104 adapted to be in contact with the tissue for debridement. As shown in the photograph 1100, a window in an encapsulation of the device 1104 provides an exposed surface 1106 of a cathode of the device 1104 for contacting the tissue for debridement. FIG. 11B shows a photograph 1110 illustrating a back side 1112 of the modified device 1104 on which a bladder 1114 (or a rubber pouch) is attached. The bladder 1114 is connected to a connector 1116 for connecting to a syringe for pumping fluid into the bladder 1114 to inflate the bladder 1114. FIG. 11C shows a photograph 1130 illustrating a side view 1132 of the modified device 1104. As shown in the FIGS. 11A to 11C, the bladder 1114 is attached and secured to the back side 1112 (i.e. the side which is not in direct contact with the tissue to be treated) of the wound debridement device. A pump (e.g. a syringe) can be connected to the bladder 1114 via the connector 1116 for inflating the bladder 1114 during use of the wound debridement device 1104. The bladder 1114 can be inflated using a fluid (e.g. air or a liquid). The bladder 1114 can be deflated after use for easy storage of the wound debridement device 1104.

The wound debridement device 1104 of the present embodiment can also be used by a patient or a bystander at home with ease. The patient or the bystander may be initially trained for using this device (or by the manual or instructions provided along with the wound debridement device 1104), and can subsequently perform wound debridement at home independently without the need to be in a clinic or a hospital. The wound debridement device 1104 can also be used under telemedicine guidance from a clinician while the patient is at home.

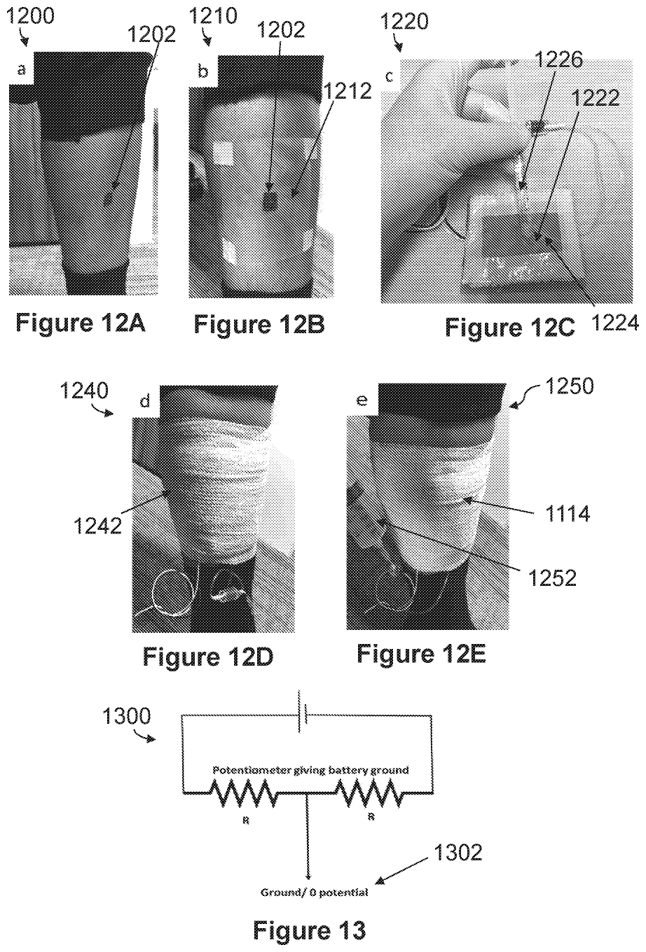

Method of application of the wound debridement device 1104 is illustrated in FIGS. 12A to 12E. In an embodiment of the wound debridement device, power can be delivered to the device through a wearable band-aid or bandage that has an integrated thin battery. Upon completion of wound debridement by the device using electrolysis, the wound can be irrigated to remove the electrolyzed debris.

FIGS. 12A, 12B, 12C, 12D and 12E show photographs illustrating steps for debriding wound tissue using the modified device 1104 of FIG. 11A in accordance with an embodiment.

FIG. 12A shows a photograph 1200 illustrating a hypothetical wound 1202 on a shin of a patient. The hypothetical wound 1202 can be covered with an insulator sheath 1212, where a cut window of the insulator sheath 1212 is placed over the hypothetical wound 1202 to expose the wound to be debrided. The insulator sheath 1212 serves therefore to protect the tissue (e.g. healthy tissue) near the wound from the tissue debridement treatment. This is shown in a photograph 1210 of FIG. 12B. FIG. 12C shows a photograph 1220 illustrating an application of an electrolyte gel 1222 on an electrode 1224 of the wound debridement device 1104. The electrolyte gel 1222 can be applied on the wound debridement device 1104 using a syringe 1226. An example of a suitable electrolyte gel includes a solution of guanidine citrate in water thickened to gel consistency by adding a solution thickening biocompatible polymer such as poly (methacrylates), poly-vinylpyrrolidine, polyvinyl alcohol, polyurethane, chitosan, sodium alginate, polyethylene glycol, or one or more of their combinations. FIG. 12D shows a photograph 1240 illustrating stabilisation of the wound debridement device 1104 over the hypothetical wound 1202 using a cotton band-aid or bandage 1242. In this case, the wound debridement device 1104 is secured over the hypothetical wound 1202 on the shin by wrapping the cotton band-aid or bandage 1242 over the wound debridement device 1104 which is placed on the shin, although it should be appreciated that other suitable securing means (e.g. an adjustable/stretchable sleeve) may also be used. Once the wound debridement device 1104 is secured over the hypothetical wound 1202 on the shin, the bladder 1114 or air pouch of the wound debridement device 1104 is inflated using e.g. a syringe 1252 to depress the electrode 1124 of the modified device 1104 onto a surface of the hypothetical wound 1202, as shown in a photograph 1250 of FIG. 12E. Although the electrode 1224, as shown in FIGS. 12A to 12E, is in direct contact with the hypothetical wound 1202, in an embodiment, an ion permeable and/or water permeable spacer can be introduced between the electrode (e.g. a cathode) and the wound surface to keep the wound from having direct contact with the electrode.

As shown in relation to FIGS. 12A to 12E, a method for debriding tissue using embodiments of the tissue debridement device therefore includes: (i) providing a suitable electrolyte (e.g. a water-based electrolyte or an electrolyte gel) to the electrolysis unit of the device, (ii) putting/placing the cathode (it may also be anode in another embodiment) which has an exposed/bare surface in contact (e.g. direct or indirect contact) with the tissue to be debrided; and (iii) connecting the two electrodes (i.e. the anode and the cathode of the electrolysis unit) to the power supply to receive the electric current for electrolysis of the electrolyte to provide the alkaline region to the tissue for debriding the tissue.

In the aforementioned embodiments for the tissue debridement device (e.g. in relation to foot exfoliation and/or wound debridement), the current supplied by the power supply to the electrolysis unit of the tissue debridement device can be controlled to regulate a pH of the alkaline region (e.g. to a pH of equal to or more than 9) for debriding the tissue. In some embodiments, a pH indicator is placed at or near the alkaline region to detect or measure a pH of the alkaline region in real time. The detected/measured pH can be used as a feedback for controlling the current supplied to the electrolysis unit for regulating or maintaining a pH of the alkaline environment for debriding the tissue.

Although the aforementioned embodiments use an alkaline environment provided by the cathode of the electrolysis unit for debriding tissue, it would be appreciated that an acidic environment provided by the anode of the electrolysis unit can also be used for debriding tissue.

FIG. 13 shows a schematic of a potentiometer circuit 1300 for providing a ground or zero potential 1302 at one of the electrodes of an electrolytic tissue debridement device in a battery powered configuration in accordance with an embodiment. The potentiometer circuit 1300 and/or the battery as shown in FIG. 13 may form part of a power supply unit for the electrolytic tissue debridement device.

### Effect of Tissue Debridement Using the Tissue Debridement Device

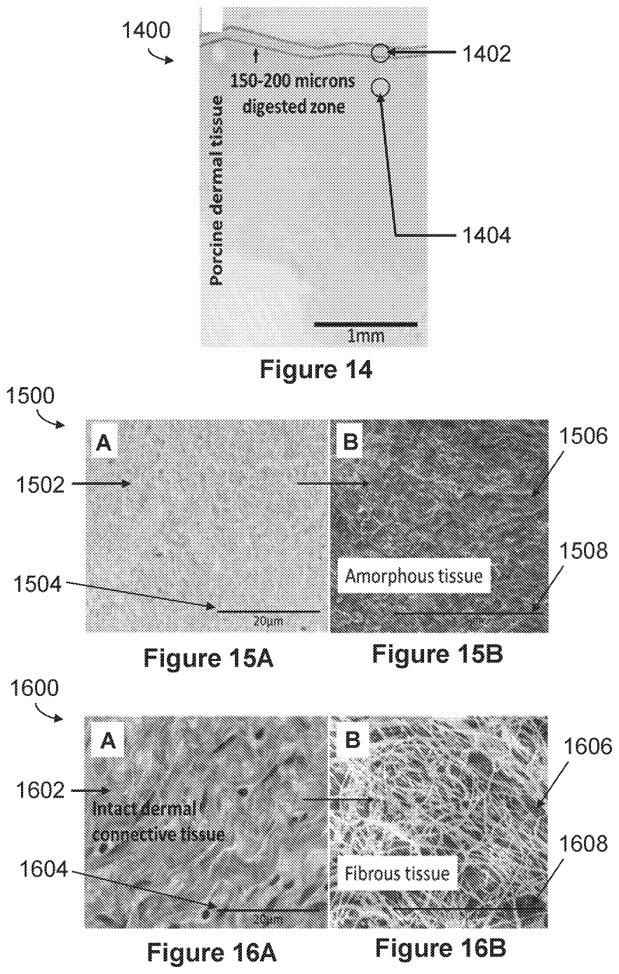

To further demonstrate an effect of tissue debridement using a tissue debridement device, the tissue debridement device of FIG. 11A was used on samples of a pig's porcine dermal tissue. The porcine dermal tissue includes dense collagenous tissues which are difficult to digest. In the present embodiment, samples of the porcine dermal tissue were exposed to the tissue debridement device for 20 minutes at a current density of 25 mA/cm² (highest current density tested). After tissue debridement using the tissue debridement device, the samples were studied under various microscopy techniques for characterisation of an effect of tissue debridement on the porcine dermal tissue. Micrographs of one of these treated samples are shown in relation to FIGS. 14 to 17 below.

FIG. 14 shows an optical micrograph 1400 of a sample of the porcine dermal tissue after surface treatment using the assembled device of FIG. 11A in accordance with an embodiment. Tissue debridement using the tissue debridement device causes denaturation and liquefaction of the tissue which is exposed to the tissue debridement device. As shown in FIG. 14, the tissue debridement treatment applied on the porcine dermal tissue produced a digested tissue zone 1402 of about 150 μm to 200 μm thick. The debridement of the porcine dermal tissue reduced a thickness of the porcine dermal tissue by about 200 μm to 350 μm, as was expected from the tissue debridement device. Micrographs in relation to this digested tissue zone 1402 and an unexposed portion 1404 within the porcine dermal tissue are shown in relation to FIGS. 15A to 16B to demonstrate an effect of tissue debridement.

FIGS. 15A and 15B show micrographs 1500 of the digested tissue zone 1402 (i.e. a surface portion of the porcine dermal tissue of FIG. 14) after surface treatment in accordance with an embodiment. FIG. 15A shows an optical micrograph 1502 of the digested tissue zone 1402 with Haematoxylin & Eosin (H&E) staining and under higher magnification. A scale bar 1504 shows a unit of 20 μm. FIG. 15B shows a micrograph 1506 of the digested tissue zone 1402 obtained using scanning electron microscopy (SEM). A scale bar 1508 shows a unit of 5 μm.

As shown in the micrographs 1502, 1506, the tissue of the surface portion becomes mostly structureless or amorphous after tissue debridement. Structural variation in the digested tissue zone 1402 as observed in the micrographs 1502, 1506 is likely in relation to this digested tissue zone 1402 being partially digested before this layer liquified during the tissue debridement process. The digested tissue zone 1402 as shown is therefore most likely to be indicative of a transition from incomplete to complete digestion as expected from the present tissue debridement process.

FIGS. 16A and 16B show micrographs 1600 of the unexposed portion 1404 of the porcine dermal tissue of FIG. 14 after surface treatment in accordance with an embodiment. FIG. 16A shows an optical micrograph 1602 of the unexposed portion 1404 with Haematoxylin & Eosin (H&E) staining with a similar magnification as the optical micrograph 1502 of FIG. 15A. A scale bar 1604 shows a unit of 20 μm. FIG. 16B shows a micrograph 1606 of the unexposed portion 1404 obtained using scanning electron microscopy (SEM). A scale bar 1608 shows a unit of 5 μm.

As shown in the micrographs 1602, 1606, the unexposed portion 1404 which is deeper within the porcine dermal tissue shows normal histology of fibrous connective tissue which is characteristic of untreated porcine dermal tissue. The micrographs 1602, 1606 together with the optical micrograph 1400 of FIG. 14 also show that after the tissue debridement treatment with the tissue debridement device, more than 95% of the thickness of the porcine dermal tissue after treatment showed normal anatomy as the untreated unexposed portion 1404.

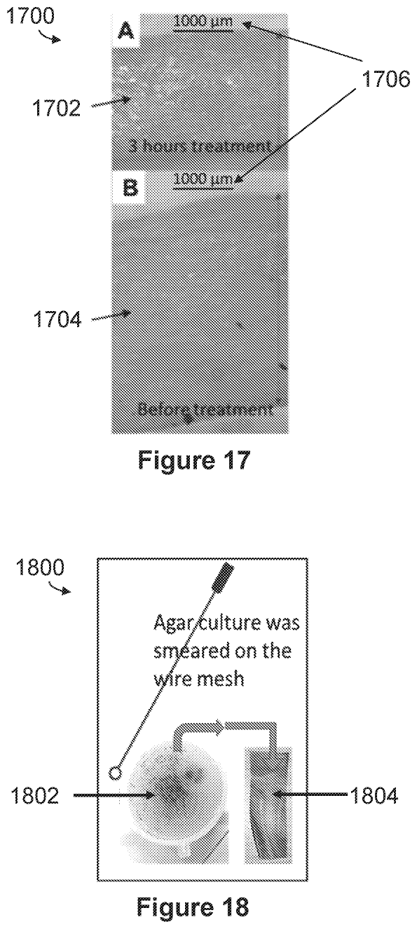

FIG. 17 shows micrographs obtained using digital microscopy to illustrate a thickness of the porcine dermal tissue of FIG. 14 before surface treatment and after surface treatment in accordance with an embodiment. The micrograph 1702 shows a thickness of the porcine dermal tissue after three hours of debridement using the tissue debridement device of FIG. 11A, and the micrograph 1704 shows an initial thickness of the porcine dermal tissue before debridement. The two micrographs 1702, 1704 are of a same scale, and scale bars 1706 of 1000 μm are shown in each of these micrographs 1702, 1704 of FIG. 17.

In the present embodiment, the thickness of the porcine dermal tissue before treatment was around 5000 microns or 5 mm. The thickness of the porcine dermal tissue after treatment was around 3000 microns or 3 mm. The current density used was 25 mA/cm². This worked out to a tissue digestive rate of approximately 10 microns per minute on the porcine dermal tissue at a current density of 25 mA/cm² for this present treatment by the tissue debridement device.

### Non-Fouling Effect of an Electrode of the Tissue Debridement Device

During electrolysis, the extreme pH on the electrode disintegrates all the organic molecular assemblies on the electrode surface. In addition, in an electrolytic system the electrical charges on the electrodes allow the formation of an ion rich hydration layer on surfaces of the electrodes. As a result of this ion rich hydration layer, ions with opposing polarity and high charge density can occupy the electrode surfaces. Ions of the same charge/polarity which are generated by the ionization of water and/or salts in the electrolyte are repelled away from the electrode surfaces. The resultant effects are that the electrodes of the tissue debridement device are adapted to repel larger particles (e.g. bacteria, microbes, and/or biological macromolecules) with low charge densities away from the electrode surfaces. This provides a non-fouling surface for the electrode of the tissue debridement device which is in contact with a wound surface during tissue debridement. In an embodiment where the cathode of the tissue debridement device provides a high pH (i.e. a pH of more than 9) region for tissue debridement, the surface of the cathode which is in contact with the wound provides a non-fouling coverage that stays clean without formation of a biofilm even in an environment comprising wound exudate. Further, the non-fouling cathode surface does not adhere to the wound surface and thus minimises recurring pain experienced during an opening and an examination of the wound bed. Microbubbles released during electrolysis also maintain a constant de-sloughing scrubbing effect on the wound surface.

FIG. 18 shows photographs of an agar culture 1802 and a wire mesh electrode 1804 to illustrate smearing of the agar culture 1802 on the wire mesh electrode surface in accordance with an embodiment.

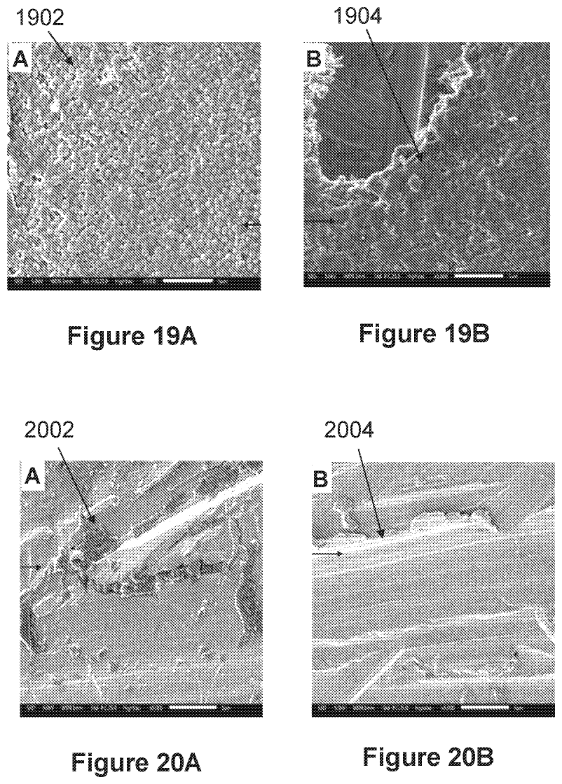

FIGS. 19A and 19B show micrographs 1902, 1904 obtained using scanning electron microscopy (SEM) in relation to the agar culture 1802 of FIG. 18. FIG. 19A shows the micrograph 1902 of the agar culture 1802 on an agar plate. The agar culture 1802 includes a confluent bacterial cocci culture formed on the agar plate. FIG. 19B shows the micrograph 1904 of the agar culture 1802 smeared on a surface of the wire mesh electrode 1804.

The sample comprising the agar culture 1802 smeared on the surface of the wire mesh electrode 1804 was divided to form two samples. One of these samples was washed in a 10% sodium dodecyl sulfate (SDS) in water, and the other of these samples was electrolysed as a cathode in a saturated sodium bicarbonate solution in water for 5 minutes and at a current density of 5 mA/cm².

FIGS. 20A and 20B show micrographs 2002, 2004 obtained using scanning electron microscopy (SEM) after treating surfaces smeared with the agar culture of FIG. 18. FIG. 20A shows the micrograph 2002 of an agar culture-smeared surface after treating with the surfactant comprising 10% sodium dodecyl sulfate (SDS), and FIG. 20B shows the micrograph 2004 of an agar culture-smeared surface after the surface is electrolysed as a cathode in a saturated sodium bicarbonate solution.

As shown in the micrograph 2002, the surfactant washed sample had a reduced bacterial load on it but still shows bacterial layers stuck to its surface. On the other hand, the micrograph 2004 of the electrolysed sample shows a clean surface. Particularly, during electrolysis, electric charge on the electrode provides for the extreme pH region on the surface of the cathode which digests all the organic molecules on the cathode surface. Further, the electric charge of the cathode attracts ions of high charge densities and forms a layer of hydration on the surface of the cathode, thereby repelling microparticles of low charge densities, including the bacteria of the agar culture, away from the electrode surface. This creates a cleaning effect on the surface of the cathode of the tissue debridement device. Moreover, the micro-gas bubbles released at the electrodes during electrolysis also contribute to a scrubbing action at the electrodes.

### Advantages of the Electrolytic Tissue Debridement Device as a Foot Exfoliation Device or a Wound Debridement Device

Foot exfoliation device—Regular exfoliation and foot hygiene is very important in preventing common foot problems like cracks, callosities and fissures. In diabetes, the foot sole eventually thickens and dry out to become brittle. A diabetic foot often starts as a contaminated crack in the sole that progresses into an infected ulcer. If the foot exfoliation and moisturizing can make the skin softer and more elastic, it may reduce precipitation of diabetic ulcers on the foot sole. Hence, the electrolytic tissue debridement device used as a foot exfoliation device may be a preventive measure for all diabetic patients to delay and possibly reduce diabetic foot conditions. This contributes to a reduction in the healthcare costs.

Unlike mechanical devices, a patient using the foot exfoliation device need not repeatedly strain to reach for his or her foot sole to perform foot scrubbing or exfoliation. The foot exfoliation device in the form of a wearable patch/pad (e.g. as shown in FIG. 9A) can effect exfoliation by electrical means without the need for repeated mechanical placements and movements. Hence, this device is suitable for old, debilitated, obese people or people with joint problems (arthritis), allowing them to perform foot exfoliation themselves. This provides a clear advantage over conventional devices and/or methods for foot exfoliation. The foot exfoliation device can also be used for cosmetics purposes.

Wound debridement device—The present wound debridement practices, although having evolved over the years, are far from their potential efficiencies. Particularly, they still require complex procedures which may involve long and frequent hospitals visits and/or high medical expenditures. This adversely affects a quality of life, work productivity, and an income or savings of a patient. The situation is worse for chronic debilitating cases involving diabetic wounds, and their resulting amputations. There is a need for more effective and faster procedures for patients, which can allow for a reduced number of hospital visits, or which can be conducted in off-clinical settings such as at a patient's residence.

Further, the extreme pH (e.g. acidic or alkaline) environment used during wound debridement neutralize microbes associated with the wound, thereby reducing wound infections. Still further, electrical charges on the electrodes allow the formation of an ion rich hydration layer on surfaces of the electrodes. This creates a non-fouling surface for the electrode which is in contact with a wound surface during tissue debridement. The non-fouling surface of the electrode does not adhere to the wound surface and thus minimises recurring pain experienced during an opening and an examination of the wound bed. In embodiments, the wound debridement device can be operated using battery power and does not require any external electric supply, making it portable and amenable for offsite use. Non-fouling surface for the electrode of the tissue debridement device which is in contact with a wound surface during tissue debridement.

In embodiments, the wound debridement device is envisaged to debride wounds of normal to medium-corrugated surface morphology. In an embodiment, power for the wound debridement device is delivered through a wearable band-aid that is integrated with a thin battery. Upon completion of electrolysis, the wound can be irrigated to remove the electrolyzed debris. The wound debridement device is highly efficient in the management of chronic wounds, allowing patients to be treated in the comfort of their home, while being completely ambulant as the wound debridement device performs the wound debridement treatment. This is clearly an advantage over current wound debridement practices. As shown in the embodiment of FIGS. 12A to 12E, the wound debridement device is an easy-to-wear disposable device. The wound debridement device can stay on the wound for a few minutes to hours to complete the electrolysis for the wound debridement treatment. Once the treatment is completed, the band-aid or bandage can be stripped and disposed, and the electrolyzed debris can be removed by a simple saline syringe irrigation.

### Reduction in Healthcare System Costs

Foot exfoliation device-Regular exfoliation and foot hygiene are very important in preventing common foot problems like cracks, callosities and fissures. In a diabetic patient, these problems are common and can precipitate wounds evolving to chronic diabetic foot ulcers. Diabetes is a very common disease in Singapore—1 in 9 Singaporeans have diabetes. There are 537 million diabetic patients worldwide in 2021. Prevalence of diabetic foot ulcer in the diabetic population is around 6.3%.

Annual incidence of diabetic foot ulcer is around 25%. Global market size of wound management products currently is around US$18.99 billion, out of which the debridement market is worth US$1,750 million (9.22%, 2017). This includes both chronic wounds (e.g., diabetic ulcers) and acute wounds (e.g., accidents, injuries). The wound management market is estimated to grow US$25 billion by 2025 of which US$3.4 billion will be the debridement market share, vastly owing to the ongoing socioeconomic development in Asia/developing nations, as well as due to the increase in global diabetes cases.

A good and effective foot exfoliation and moisturizing technology has a preventive role in diabetic foot conditions. This drastically reduces the expenses on chronic wound/ulcer management and thus contributes to significant reduction in the healthcare costs.

Wound debridement device—With training, the diabetic foot ulcer patients or their caretakers can use the wound debridement device at home, with or without guidance from a clinician online through a telemedicine system. This reduces the cost of wound care of the chronic diabetic foot ulcer patients by reducing the number of visits to the hospitals and hospital admissions.

### Demand for Foot Exfoliation Device

The demand for foot care products is on a surge mainly due to the following factors: (i) alarming levels of diabetes incidence worldwide, (ii) incidence of overweight, (iii) emerging middle class in developing countries, (iv) rising interest in grooming among men signals opportunities, and (v) growing interest in self-care and over-the-counter (OTC) foot care products. The main target for the foot exfoliation device is diabetic patients who are vulnerable to develop foot ulcers if they ignore foot exfoliation and hygiene. There are 537 million diabetic patients in 2021. Globally around 9.3% of people are diabetic. Hence, the diabetic population needs regular foot exfoliation and moisturizing. Besides this, on the cosmetic front, the global cosmetic foot scrub market size in 2020 alone was US$343 million. There is therefore huge potential and demand for an improved foot exfoliation device, such as the one described in the present disclosure.

### Demand for Wound Debridement Device

On average, 10% of the global diabetic patients develop diabetic foot ulcerations (chronic wounds on the feet) in their life time and such foot ulcerations typically take several months to heal. This put the global diabetic foot ulcer prevalence at more than 50 million. These cases at times need wound debridement daily, or alternate days, or once in a week, depending on their stage of healing or any complications that they have. Assuming this population would require wound dressing once in 3 days, this demands 17 million wound dressings performed daily for diabetic foot ulcer cases alone. This is excluding other major causes of wounds like road traffic accidents, trauma and burns. A diabetic foot wound debridement process in Singapore clinics costs at least S$100 per debridement session, bringing the daily investment on global chronic diabetic foot ulcer care to around S$2 billion.

It should be appreciated that although embodiments as described include a foot exfoliation device, a wound debridement device and a tissue debridement device, all these embodiments work on a same principle of using a non-neutral pH environment (i.e. pH away from pH=7, e.g. an acidic or an alkaline environment) to remove or debride unwanted tissue. They may differ in relation to an anatomical location at which the device is used and/or a depth of tissue removal required.

Alternative embodiments of the device for debriding tissue include: (i) another one of the two electrodes (i.e. the electrode which is not used for providing a non-neutral pH (e.g. an acidic or alkaline environment/region) for debriding the tissue) which is grounded (an example of a circuit for providing ground or zero potential for the another one of the two electrodes using a battery powered configuration is shown at FIG. 13); (ii) the electrolysis unit comprising an encapsulation made of an electrically insulating material for receiving the electrolyte, the encapsulation having at least a window for exposing an anode or a cathode to the tissue for providing a respective acidic or alkaline region during electrolysis of the electrolyte for debriding the tissue; (iii) a solid electrolyte or an electrolyte gel; (iv) a water-based electrolyte comprising an organic salt or an inorganic salt, the organic salt or the inorganic salt having a concentration of between 1 molar to 5 molar, examples of suitable organic salt includes: (1) guanidine citrate, (2) guanidine ascorbate, (3) guanidine carbonate, (4) amino-acid salts, (5) creatine salts, (6) acetic acid salts, (7) lactic acid salts, (8) citric acid salts, (9) ethylenediaminetetraacetic (EDTA) salt, (10) ethylene glycol-bis(β-aminoethyl ether)-N,N,N,N-tetra acetic acid tetrasodium (EGTA) salt, and (11) a complex of an amine and carboxylic acid, while examples of suitable inorganic salt includes: (1) sodium bicarbonate, (2) potassium bicarbonate, (3) sodium hydroxide, (4) potassium hydroxide, (5) calcium hydroxide, (6) potassium permanganate, (7) sodium iodide, (8) potassium iodide, (9) sodium sulfate, (10) potassium sulfate, (11) sodium phosphate, and (12) potassium phosphate; (v) the electrolyte comprising a solvent (e.g. water), a salt (e.g. an organic salt or an inorganic salt as aforementioned described), a humectant (e.g. glycerol, glycol, carbohydrate, panthenol etc.), biological molecules (e.g. tissue digesting enzymes such as papain or a healing promoting growth factor), a surfactant (e.g. sodium dodecyl sulfate (SDS), polysorbate etc.), a wetting agent (e.g. a surfactant or an emulsifier), an antacid (e.g. sodium bicarbonate which is capable of reducing acidity at an anode), an anti-foaming agent (e.g. silica dimethicone silylate like compounds that reduce build-up of bubbles between the electrodes which increases a resistance in the electrolytic device), a conditioner compound (e.g. a molecule or a compound in an exfoliation formulation which increases a smoothness, shine and/or moisture of the skin/tissue), an antibiotic (e.g. neomycin, polymyxin b etc.), an antimicrobial (e.g. an antibacterial/antiviral/antifungal/antiseptic compound such as providone iodine), a metal chelating compound (e.g. EDTA, EGTA to remove any leaching ions from metallic electrodes), a coloring compound (e.g. a superficial skin surface colouring compound such as henna) or a hydrophobic compound (e.g. oil molecules for preventing water loss from the exfoliation treated skin/tissue); (vi) a power supply comprising one of: a DC power supply, a battery or an AC-DC adaptor connected to an AC power supply; (vii) the power supply comprising a battery which is integrated with the device, or integrated with a dressing or a bandage for securing the device to the tissue for debriding; (viii) the power supply comprising a mechanism for reversing a polarity of the electric current. The mechanism for reversing a polarity of the electric current (and therefore a polarity of the electrodes) helps to terminate any debriding effect of an alkaline zone (if a cathode is used as the debriding surface) or an acidic zone (if an anode is used as the debriding surface) which is exposed to the tissue to be treated. This helps to terminate the debriding process by neutralizing the pH zone at which the tissue is exposed to, and to ensure that little or no residual alkaline or acidic zone remain on the surface of the treated tissue. Switching the polarity of the cathode for a short time before the termination of the debriding treatment therefore helps to neutralize the residual pH effect on the surface of the tissue to be treated; (ix) the power supply configured to provide an electric current having a magnitude of less than or equal to 5 A, or less than or equal to 1 A, which may be achieved by adjusting an output current or an output voltage or an output power of the power supply; (x) the power supply configured to provide an electric potential difference of less than 20 V or less than 5 V between the two electrodes; (xi) a tissue condition sensor configured to detect a condition of the tissue prior to receiving the electric current for electrolysis of the electrolyte; (xii) a treatment end-point sensor configured to determine an end-point for debriding the tissue, an example of a treatment end-point sensor includes a resistance measurement sensing circuit which can be used to detect a drop in impedance or resistance contributed by dead skin (e.g. in the case of an exfoliation device) or dead debris/tissue (in the case of a wound debridement device) once layers of the dead skin or dead tissue (dead skin or dead tissue layer lacking blood flow and nerves generally has a high resistance) are thinned out or removed, or to determine an end-point when a detected impedance or resistance matches that of a live skin/tissue. The impedance or resistance can be measured across the surface being treated, i.e. across the tissue layer being debrided/removed; (xiii) a performance sensor configured to assess a performance of the device for debriding the tissue, an example of a performance sensor includes: a pH measurement sensor for detecting a pH environment produced by the device, an ammeter for measuring a current flowing between the electrodes or a heat sensor or a temperature sensor for detecting an amount of heat or a temperature increase generated by the device; (xiv) a pH indicator configured to detect a pH of the acidic region or the alkaline region in contact with the tissue for debriding the tissue; (xv) a device for monitoring a condition of the tissue (e.g. during treatment), an example of a sensor for monitoring a condition of the tissue includes a camera for visualizing an appearance of the tissue, or an ultraviolet excitation source and visible emission detector for detecting fluorescent bacterial infection of the tissue, or an amine sensor for detecting putrefaction of the tissue; (xvi) a vibrator (e.g. a vibration motor) for providing vibration to a surface of the tissue to be treated and/or a rotator (e.g. a DC motor), where the vibrator and/or the rotator are configured to provide mechanical friction (e.g. by vibration or rotation) between a surface of the electrode and a surface of the tissue being treated. The vibrator may be attached on an external side of the electrolysis unit (i.e. on an opposite side to the tissue to be treated); (xvii) an irrigator or an electrolyte infuser, the irrigator or the electrolyte infuser can either be manual or electronic in nature, and the irrigator or the electrolyte infuser is configured to provide irrigation of electrolyte from a reservoir either to sustain electrolysis or to wash away a debriding surface of the tissue/wound. The irrigator or the electrolyte infuser may be connected to the electrolysis unit; (xviii) one or both of the electrodes comprising a wire mesh or a conductive felt or a conductive plate/sheet. A wire mesh may concentrate charged ions at a higher density than a conductive felt/plate/sheet (due to a reduced surface area of the wire mesh compares to the conductive felt/plate/sheet) and may be preferred in circumstances where a higher density of ions is desired. In embodiments, the conductive felt/plate/sheet and/or the wire mesh are resistant to corrosion due to electrolysis by the electrolyte in the tissue debriding device; (xix) a pressure management system which is adapted to vary (e.g. increase or decrease) a fluid pressure of the electrolyte between the electrodes. The fluid pressure (positive or negative) of the electrolyte exerting on the electrodes can be transferred to the tissue to be treated through the electrode which is in contact with the tissue. The pressure management system can be used in conjunction with a securing means (e.g. the cotton band-aid or bandage as shown in relation to FIG. 12D used to secure or stabilise a tissue debridement device). In an embodiment comprising a perforated electrode, negative pressure applied to the electrolyte may result in suction of fluid from the perforated electrode. This may provide a suction force on the tissue to be treated for putting the perforated electrode in contact with the tissue. In an embodiment, the pressure management system includes a pressure sensor configured to measure a pressure associated with the electrolysis unit of the tissue debridement device and/or a pressure exerted on the tissue/wound to be treated by the electrode of the tissue debridement device; (xx) a kit of parts arranged to be assembled to form a tissue debridement device, the kit of parts comprising a conductive sheet/plate (e.g. a graphite sheet or a flexible graphite sheet), a conductive porous sheet/plate (e.g. a steel wire mesh). In embodiments where the electrolyte is water-based, the kit of parts may comprise an absorbent material (e.g. a cotton band aid). The absorbent material may not be necessary if the electrolyte used is e.g. a solid electrolyte; (xxi) a thin ion permeable spacer layer between an electrode surface (i.e. the electrode surface to be used to treat the tissue) and the tissue to be treated. In this case, the electrode treating the tissue is in indirect contact with the tissue; and (xxii) a system for debriding tissue comprising one of the aforementioned tissue debridement devices and one or more aforementioned sensors/systems (e.g. a pressure management system, a tissue condition sensor, a pH sensor/indictor, a performance sensor etc.).

Although only certain embodiments of the present invention have been described in detail, many variations are possible in accordance with the appended claims. For example, features described in relation to one embodiment may be incorporated into one or more other embodiments and vice versa.

1. A device for debriding tissue, the device comprising:
an electrolysis unit having two electrodes, the electrolysis unit being configured to receive an electrolyte for electrically connecting the two electrodes, the two electrodes are adapted to connect to a power supply to receive an electric current for electrolysis of the electrolyte,
wherein in use, one of the two electrodes is adapted to provide an acidic region or an alkaline region to the tissue during electrolysis of the electrolyte for debriding the tissue.

2. The device of claim 1, wherein the one of the two electrodes is perforated or includes a wire mesh, the one of the two electrodes is adapted to allow ions having a same polarity as the one of the two electrodes to pass through to an outer side of the electrolysis unit during electrolysis of the electrolyte to provide the acidic region or the alkaline region for debriding the tissue.

3. The device of claim 1, wherein the electrolysis unit comprises an encapsulation made of an electrically insulating material for receiving the electrolyte, the encapsulation having a window for exposing the one of the two electrodes to the tissue for providing the acidic or alkaline region during electrolysis of the electrolyte.

4. The device of claim 1, further comprising the electrolyte received by the electrolysis unit.

5. The device of claim 4, wherein the electrolyte is water-based, the one of the two electrodes is adapted to form an ion-rich hydration layer on an electrode surface of the one of the two electrodes during electrolysis of the electrolyte, the ion-rich hydration layer being adapted to repel ions having a same polarity as the one of the two electrodes away from the electrode surface or particles having a mass equal to or less than 1000 daltons and carrying at least an elementary charge away from the electrode surface.

6. The device of claim 4, wherein the electrolyte includes an organic salt or an inorganic salt, the organic salt or the inorganic salt has a concentration of between 1 molar to 5 molar, or wherein the electrolyte includes a solvent, a salt, a humectant, biological molecules, a surfactant, a wetting agent, an antacid, a pH buffering formulation, an anti-foaming agent, a conditioner compound, an antibiotic, an antimicrobial, a metal chelating compound, a coloring compound or a hydrophobic compound.

7. The device of claim 1, wherein the one of the two electrodes is a cathode, the cathode is adapted to provide the alkaline region having a pH level of 9 or above during electrolysis of the electrolyte for debriding the tissue.

8. The device of claim 1, further comprising the power supply, the power supply comprising a mechanism for reversing a polarity of the electric current.

9. The device of claim 1, further comprising a tissue condition sensor configured to detect a condition of the tissue prior to receiving the electric current for electrolysis of the electrolyte or a treatment end-point sensor configured to determine an end-point for debriding the tissue.

10. The device of claim 1, further comprising a performance sensor configured to assess a performance of the device for debriding the tissue or a pH indicator configured to detect a pH of the acidic region or the alkaline region in contact with the tissue for debriding the tissue.

11. The device of claim 1, further comprising a vibrator, a rotator, an irrigator, an electrolyte infuser, or a pressure management system adapted to vary a fluid pressure of the electrolyte between the two electrodes.

12. The device of claim 1, further comprising an inflatable bladder attached to the electrolysis unit at an opposite side to the one of the two electrodes, the inflatable bladder being configured to inflate so as to apply pressure on the electrolysis unit for engaging the one of the two electrodes on the tissue when the device is secured on the tissue.

13. The device of claim 1, wherein the tissue includes skin tissue or wound tissue.

14. A kit of parts arranged to be assembled to form the device of claim 1, wherein the kit of parts comprises a graphite sheet and a conductive porous sheet for use as the electrodes of the electrolysis unit and a non-conductive material for use as a spacer between the electrodes of the electrolysis unit.

15. A method for debriding tissue using a device, the device comprising an electrolysis unit having two electrodes, the electrolysis unit is configured to receive an electrolyte for electrically connecting the two electrodes, the two electrodes are adapted to connect to a power supply to receive an electric current for electrolysis of the electrolyte, wherein one of the two electrodes is adapted to provide an acidic region or an alkaline region to the tissue during electrolysis of the electrolyte for debriding the tissue, the method comprising:
providing the electrolyte to the electrolysis unit;
putting the one of the two electrodes in contact with the tissue; and
connecting the two electrodes to the power supply to receive the electric current for electrolysis of the electrolyte to provide the acidic region or the alkaline region to the tissue for debriding the tissue.

16. The method of claim 15, further comprising controlling the electric current to regulate a pH of the acidic region or the alkaline region for debriding the tissue.

17. The method of claim 15, wherein the one of the two electrodes is a cathode, the method further comprises controlling the electric current to provide the alkaline region having a pH level of 9 or above at the cathode during electrolysis of the electrolyte for debriding the tissue.

18. The method of claim 15, wherein putting the one of the two electrodes in contact with the tissue includes securing the device to the tissue using a wound dressing or an adhesive bandage, or generating a suction force at the one of the two electrodes by applying a negative fluid pressure on the electrolyte.

19. The method of claim 15, further comprising providing a solvent, a salt, a humectant, biological molecules, a surfactant, a wetting agent, an antacid, an anti-foaming agent, a pH buffering formulation, a conditioner compound, an antibiotic, an antimicrobial, a metal chelating compound, a coloring compound or a hydrophobic compound in the electrolyte.

20. The method of claim 19, wherein putting the one of the two electrodes in contact with the tissue exposes the tissue to the acidic or alkaline region during electrolysis of the electrolyte for relaxing a network of the tissue so as to allow permeation of the humectant, the biological molecules, the surfactant, the wetting agent, the antacid, the anti-foaming agent, the pH buffering formulation, the conditioner compound, the antibiotic, the antimicrobial, the metal chelating compound, the coloring compound or the hydrophobic compound to penetrate into the tissue.
